(19) United States
(12) Patent Application Publication
(10) Pub. No.: US 2018/0279911 A1
(43) Pub. Date: Oct. 4, 2018

(54) ANALYTE SENSOR USER INTERFACE APPARATUS AND METHODS

(71) Applicant: GlySens Incorporated, San Diego, CA (US)

(72) Inventors: Joseph Lucisano, San Diego, CA (US); Timothy Routh, San Diego, CA (US); Michael Perkins, Los Gatos, CA (US); Kamyar Keshmiri, San Francisco, CA (US); Adam Cristobal, San Francisco, CA (US)

(21) Appl. No.: 15/472,091

(22) Filed: Mar. 28, 2017

(51) Int. Cl. A61B 5/07; A61B 5/00; A61B 5/145; A61B 5/1473; A61B 5/1486; G08B 5/22; G08B 21/18; G08B 6/00; G08B 3/10

(52) U.S. Cl. CPC A61B 5/076; A61B 5/0031; A61B 5/14532; A61B 5/14542; A61B 5/1473; G08B 3/10; A61B 5/4839; A61B 5/7475; G08B 5/22; G08B 21/182; G08B 6/00; A61B 5/14865

(57) ABSTRACT

User interface (UI) apparatus and associated logic and methods for use with a blood analyte sensor. In one embodiment, the blood analyte sensor comprises an implantable blood glucose sensor, and the UI and associated logic are configured to receive and store user-specified parameters for operation of the blood analyte sensor and/or associated receiver. In one exemplary implementation, the UI and associated logic are configure to selectively implement various user interface regimes, including display of various analyses of current blood analyte level data and/or historical blood analyte level data, as well as prompting the user to take actions. The exemplary implementation is also optionally configured to identify and mitigate data errors.

### RELATED APPLICATIONS

This application is related to co-owned and co-pending U.S. patent application Ser. Nos. 13/559,475 filed Jul. 26, 2012 entitled “Tissue Implantable Sensor With Hermetically Sealed Housing,” 14/982,346 filed Dec. 29, 2015 and entitled “Implantable Sensor Apparatus and Methods”, 15/170,571 filed Jun. 1, 2016 and entitled “Biocompatible Implantable Sensor Apparatus And Methods”, 15/197,104 filed Jun. 29, 2016 and entitled “Bio-adaptable Implantable Sensor Apparatus And Methods”, 15/359,406 filed Nov. 22, 2016 and entitled “Heterogeneous Analyte Sensor Apparatus and Methods”, and 15/368,436 filed Dec. 2, 2016 and entitled “Analyte Sensor Receiver Apparatus and Methods”, each of the foregoing incorporated herein by reference in its entirety. This application is also related to U.S. patent application Ser. No. 10/719,541 filed Nov. 20, 2003, now issued as U.S. Pat. No. 7,336,984 and entitled “Membrane and Electrode Structure for Implantable Sensor,” also incorporated herein by reference in its entirety.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### 1. Technical Field

The disclosure relates generally to the field of sensors, therapy devices, implants and other devices (such as those which can be used consistent with human beings or other living entities for in vivo detection and measurement or delivery of various solutes), and in one exemplary aspect to methods and apparatus enabling the use of such sensors and/or electronic devices for, e.g. monitoring of one or more physiological parameters, including through use of a user interface (UI) associated with an external receiver and processing apparatus.

### 2. Description of Related Technology

Implantable electronics is a rapidly expanding discipline within the medical arts. Owing in part to significant advances in electronics and wireless technology integration, miniaturization, performance, and material biocompatibility, sensors or other types of electronics which once were beyond the realm of reasonable use in vivo in a living subject can now be surgically implanted within such subjects with minimal effect on the recipient subject, and in fact many inherent benefits.

One particular area of note relates to blood analyte monitoring for subjects, such as for example glucose monitoring for those with so-called “type 1” or “type 2” diabetes. As is well known, regulation of blood glucose is impaired in people with diabetes by: (1) the inability of the pancreas to adequately produce the glucose-regulating hormone insulin; (2) the insensitivity of various tissues that use insulin to take up glucose; or (3) a combination of both of these phenomena. Safe and effective correction of this dysregulation requires blood glucose monitoring.

Currently, glucose monitoring in the diabetic population is based largely on collecting blood by “fingersticking” and determining its glucose concentration by conventional assay. This procedure has several disadvantages, including: (1) the discomfort associated with the procedure, which should be performed repeatedly each day; (2) the near impossibility of sufficiently frequent sampling (some blood glucose excursions require sampling every 20 minutes, or more frequently, to accurately treat); and (3) the requirement that the user initiate blood collection, which precludes warning strategies that rely on automatic early detection. Using the extant fingersticking procedure, the frequent sampling regimen that would be most medically beneficial cannot be realistically expected of even the most committed patients, and automatic sampling, which would be especially useful during periods of sleep, is not available.

Implantable glucose sensors have long been considered as an alternative to intermittent monitoring of blood glucose levels by the fingerstick method of sample collection. These devices may be fully implanted, where all components of the system reside within the body and there are no through-the-skin (i.e. percutaneous) elements, or they may be partially implanted, where certain components reside within the body but are physically connected to additional components external to the body via one or more percutaneous elements. Fully implanted sensors that do not contain a power source also require wearing on the skin, in close proximity to the sensor, an external device to wirelessly (for example by inductive coupling) convey power to the sensor and communicate with the sensor to receive data. Fully implanted sensors that do contain a power source need not require the wearing of any accessory device on the skin, as they may be configured to transmit sensor data to a receiver some distance away.

FIG. 1 is a block diagram illustrating one specific exemplary prior art percutaneous blood glucose monitoring system 100. Exemplary of this class of devices is the Dexcom G5® Mobile CGM System. Manufactured by Dexcom Corporation, the Dexcom G5 system comprises a device 102 worn on the subject's outer abdomen 101, the device 102 which includes a small, transcutaneous probe (sensor needle) 103 with peroxide-based detector element 105, and detector circuitry 104 with wireless transmitter (i.e., Bluetooth-enabled device) 106. The transmitter 106 communicates wirelessly with a receiver/display device 108 which can be either (i) a user's smartphone or other personal electronic device with the Dexcom software “app” installed thereon, or (ii) a Dexcom G5 mobile receiver device (i.e., dedicated receiver) for display of a user interface (UI). With the Dexcom G5 system, the user has access (via interface of the receiver or smartphone running the mobile app to a LAN/WAN 121) to a cloud-based reporting system (Dexcom CLARITY®), which ostensibly enables access and tracking of the user's glucose data via a web-based platform.

Devices such as the Dexcom G5 utilize peroxide-based blood glucose sensing, including a requirement for frequent external calibration (i.e., utilizing a separate confirmatory test such as a fingerstick). Per the manufacturer, the device should be calibrated at least once every twelve (12) hours via fingerstick or blood glucose (BG) meter, thereby making the device somewhat burdensome for the user in everyday operation (aside from issues associated with the transcutaneous sensor probe 103 and the associated requirement to continuously wear the device 102 externally on the abdomen).

For the exemplary Dexcom G5 system, user interface (UI) displays at e.g., the receiver/display device 108 or other device (see FIGS. 1A and 1B) may include a current blood glucose reading 150, an indicator 152 showing whether the blood glucose level is rising or falling, and a trend graph of blood glucose concentration over time 154 showing a high glucose alert level 156 and a low glucose alert level 158. As shown in FIG. 1B, the foregoing data can be displayed at another person's smartphone or other personal electronic device with the Dexcom software “app” installed thereon (i.e., a device associated with a person other than the user).

The Assignee hereof has more recently developed improved methods and apparatus for implanting and measuring blood glucose level which overcome the aforementioned disabilities (as well as other disabilities) with the prior art; see, inter alia, U.S. patent application Ser. Nos. 13/559,475, 14/982,346, 15/170,571, 15/197,104, 15/359,406 and 15/368,436 previously incorporated herein. However, the Assignee has further recognized that areas of potential improvement over the prior art relate to, inter alia, the UI for the external device (e.g., a receiver or another device) associated with an implanted sensor, as well as other modalities of interacting with the implanted sensor and/or data produced thereby.

For example, in conventional sensors such as, e.g., the transcutaneous Dexcom G5 system discussed supra, the UI may provide alerts to a user only when a detected analyte concentration has reached a level which is prospectively dangerous to the user's health (e.g., blood glucose falls below the low glucose alert level 158 or rises above a high glucose alert level 156). In some instances, due to latency in the system and other factors, the alert may be delivered to the user after a period when the user is able to respond to the alert and self-manage the condition, thereby risking the patient's health and/or requiring health professional intervention. Such techniques are almost necessarily reactive in nature; only when the alert level is reached is action taken by the software to alert the user. Unless the user is closely monitoring the trend 152 and/or graph 154, they may simply be alerted after a potentially dangerous blood glucose level has occurred. Alerts may also be issued based on, inter alia, the need for repeated device calibration, or reduced accuracy (e.g., over/under-sensitivity to analyte concentration, one or more non-functional sensor elements, etc.). Repeated alerts may also be issued for a single analyte concentration-related event. Such frequent alerting can be disruptive to the user such as e.g., when sleeping or in a public location, and may also lead to a level of frustration, and even desensitization of the user over time to such alerts.

Such alert functionality does not take any context relating to the user or their then-prevailing individual situation or condition into account in monitoring for and delivering alerts. For instance, the timing and manner in which an alert is delivered when the user is in one context (e.g., awake and ambulatory) may be inappropriate for another context (such as when the user is sleeping).

Moreover, such blood analyte detection systems do not allow for personalization of notification delivery, which is desirable in that disease presentation and lifestyle may be different for each user, and may also change as a function of time.

Accordingly, there is a salient need for more “intelligent” and user-adaptable methods and apparatus for interfacing such analyte sensing systems with the user.

### SUMMARY

The present disclosure satisfies the foregoing needs by providing, inter alia, improved apparatus including a user interface (UI) and associated logic and methods, for accurately providing information relating to sensed analyte levels within a living subject, including for extended periods of time without explant.

In one aspect of the disclosure, an apparatus for use with an implantable blood analyte sensing device is disclosed. In one embodiment, the apparatus includes a display device and a data processing apparatus in data communication with the display device, the data processing apparatus having a computer program stored thereon, which when executed, causes the data processing apparatus to: (i) receive blood analyte data from a receiver apparatus configured for signal communication with an implanted analyte sensor; (ii) process the blood analyte data to determine at least a blood analyte concentration; and (iii) generate a user interface (UI) for display on the display device.

In one variant, the implanted analyte sensor is a glucose sensor and the blood analyte concentration is a blood glucose concentration. In one implementation, the glucose sensor is an oxygen-based glucose sensor. In another implementation, the glucose sensor is a hydrogen peroxide-based glucose sensor. In yet another implementation, the glucose sensor is a hydrogen peroxide-based and oxygen-based glucose sensor.

In another variant, the receiver apparatus is configured for wireless signal communication with the implanted analyte sensor. In one implementation, the data processing apparatus and the receiver apparatus comprise a receiving and processing device having the display device disposed therein. In another implementation, the data processing apparatus is an Internet Protocol (IP)-enabled device having the display device disposed therein and the receiver apparatus is a user wearable device. In one example, the IP-enabled device is a user mobile device. In another example, the IP-enabled device is a user computing system.

In another embodiment, the data processing apparatus is further configured to receive user input including user preferences and/or parameters via the UI. In one variant, the user preferences and/or parameters include an analyte concentration upper limit, an analyte concentration pre-warning upper limit, an analyte concentration lower limit, and/or an analyte concentration pre-warning lower limit. In one implementation, each of the foregoing analyte concentration limits are associated with a specific time period and/or a specific day of the week input by the user.

In another implementation, the analyte concentration pre-warning upper limit and the analyte concentration pre-warning lower limit comprise a pre-warning buffer zone displayable in the UI, while the analyte concentration upper limit and the analyte concentration lower limit comprise a danger zone displayable in the UI. In one example, the buffer zone and the danger zone are displayable in an overlaid configuration on the UI, and a current blood analyte concentration and/or blood analyte concentration trend data are graphically displayable relative to the buffer and danger zones.

In a further embodiment, the computer program, when executed, further causes the data processing apparatus to provide alerts to the user via a user alert mechanism. In one variant, a soft alert is provided in response to a current blood analyte concentration being above an analyte concentration pre-warning upper limit and/or below an analyte concentration pre-warning lower limit.

In another variant, a priority alert is provided in response to a current blood analyte concentration being above an analyte concentration upper limit and/or below an analyte concentration lower limit.

In yet another variant, the alerts are one or more of an audible alert, a haptic alert, or a visual alert provided via the user alert mechanism. In one implementation, the user alert mechanism and the processing apparatus are a wearable device. In another implementation, the user alert mechanism is a wearable device separate from the processing apparatus.

In another embodiment, the computer program, when executed, further causes the data processing apparatus to provide alerts to a caretaker via a caretaker alert mechanism. In one variant, a priority alert is provided in response to (i) a current blood analyte concentration being above an analyte concentration upper limit and/or below an analyte concentration lower limit and (ii) a user response time being beyond a predetermined time limit.

In another variant, the alerts are one or more of an audible alert, a haptic alert, or a visual alert provided via the caretaker alert mechanism. In one implementation, the caretaker alert mechanism and the processing apparatus are a wearable device. In another implementation, the caretaker alert mechanism is a wearable device separate from the processing apparatus.

In an additional embodiment, the computer program, when executed, further causes the data processing apparatus to receive user action input in response to an alert. In one variant, a selectable list of user actions is displayed in the UI. In one implementation, the selectable list of user actions includes a selectable icon for entering a manual analyte concentration reading and/or a selectable icon for intake of medication. In another implementation, the selectable list of user actions includes a selectable icon for intake of fast-acting carbohydrates and/or a selectable icon for intake of slow acting carbohydrates.

In another variant, based at least on the received user action input, analyte concentration over time is graphically displayed on the UI and an icon for received user action input is overlaid on the graphical display.

In yet another variant, the computer program, when executed, further causes the data processing apparatus to provide warning alerts to the user via a user alert mechanism in response to determination of a blood analyte concentration event. Furthermore, based at least on the received user action input, a delay period is invoked prior to provision of subsequent alerts for the blood analyte concentration event.

In yet another embodiment, the computer program, when executed, further causes the data processing apparatus to display blood analyte concentration data over a period of time. In one variant, a duration of the period of time is user selectable.

In another variant, the blood analyte concentration data over the period of time includes at least a first selectable graphical analysis and a second selectable graphical analysis (i.e., different from the first graphical analysis). In another aspect of the disclosure, a method of operating an apparatus for use with an implantable blood analyte sensing device is disclosed. In one embodiment, the method includes receiving, at a processing apparatus, blood analyte data from a receiver apparatus configured for signal communication with an implanted analyte sensor; processing the blood analyte data to determine at least a blood analyte concentration; and generating a UI for display of at least the blood analyte concentration on a display device in data communication with the processing apparatus.

In another aspect of the disclosure, a method of monitoring a blood analyte level of a living being using a blood analyte sensing apparatus is disclosed. In one embodiment, the method includes: receiving blood analyte data from a blood analyte sensing apparatus; evaluating the received data, the evaluating comprising calculating at least one parameter; and based at least on the at least one parameter, configuring at least one user interface alert function.

In one variant, the received blood analyte data are generated periodically by a fully implanted oxygen-based blood glucose sensing apparatus, and the calculating comprises calculating: (i) rate of change of blood glucose level; and (ii) blood glucose level. In one implementation, the configuring at least one user interface alert function comprises establishing upper and lower buffer zones within which only soft alerts to the user are provided. For instance, in one such implementation, the range of the lower zone is smaller than the range of the upper zone, and the method further comprises alerting the user more rapidly for a given rate of change (ROC) when in the lower zone than in the upper zone, so as to more rapidly alert the user to the possibility of a hypoglycemic event.

In another implementation, the provision of soft alerts comprises providing only non-audible alerts selected from the group consisting of (i) haptic alerts via a haptic apparatus of a receiver worn by the user, and (ii) visual alerts on a display device of the receiver.

In another variant, the calculating comprises calculating: (i) rate of change of blood analyte level; and (ii) blood analyte level; the configuring at least one user interface alert function comprises establishing upper and lower buffer zones within which only soft alerts to the user are provided, and the method further includes providing hard alerts when the blood analyte level falls outside of either the upper or lower buffer zones.

In another embodiment, the method further includes receiving data from a sensor apparatus, the data indicative of at least one user context; evaluating the data using a computerized process to identify a context; the establishment of the upper and lower buffer zones includes establishing at least one of the upper and lower zones based at least on the identified context.

Moreover, in one variant, the method further includes: accessing data indicative of a most recent calibration event; calculating a value using at least (i) the accessed data indicative of a most recent calibration event and (ii) data indicative of at least one of a current date and/or current time, the calculated value indicative of at least one of a date difference and/or time difference; and establishing at least one of the upper and lower zones based at least on the identified context and the calculated value.

In another aspect, apparatus for use with an implanted blood analyte sensing device is disclosed. In one embodiment, the apparatus includes: wireless receiver apparatus configured to receive wireless signals from the blood analyte sensing device, the wireless signals encoding data relating to levels of said blood analyte; data processing apparatus in data communication with the wireless receiver apparatus and configured to utilize said encoded data to determine a blood analyte level; an electrical power source configured to supply electrical power; indicator apparatus in communication with the data processing apparatus and electrical power source, the indicator apparatus configured to indicate to a user the determined blood analyte level; and at least one computer program operative to run on the data processing apparatus and configured to selectively determine at least one of an upper buffer zone and lower buffer zone, the at least one buffer zone comprising a range of blood analyte level values within which a first alert regime is applied, and outside of which at least a second alert regime is applied, the second alert regime configured to be more intrusive to the user than the first regime.

In one variant, the implanted blood analyte sensing device comprises at least one oxygen-based sensor, and said apparatus is configured to operate without communication with a parent platform or external calibration for at least one (1) week.

In a further aspect, a method of monitoring a blood analyte level of a living being using a blood analyte sensing apparatus is disclosed. In one embodiment, the method includes determining a context of the user, and based on the determined context, implementing a context-specific monitoring and alert regime.

In one variant, the determining a context comprises at least: receiving data from an accelerometer apparatus of the blood analyte sensing apparatus; evaluating the received data to identify one or more movement patterns associated with a user behavior; and based at least on the identification of the one or more patterns, determining the context. In one implementation, the identification of the one or more movement patterns comprises comparison of at least a portion of the received data to one or more pre-stored templates.

In another variant, the blood analyte sensing apparatus comprises: (i) an implanted blood analyte sensor and (ii) a local receiver apparatus in wireless communication with the implanted sensor; and the determining a context comprises at least using a personal assistant application program of a computerized device with which the local receiver apparatus is in data communication to access a network-based application programming interface (API) to obtain context data, the context data used in the determining the context.

In another aspect, a method of monitoring a blood analyte level of a living being using a blood analyte sensing apparatus comprising an implanted sensor apparatus and a receiver apparatus is disclosed. In one embodiment, the method includes: receiving at the receiver apparatus blood analyte data from the implanted sensor apparatus; evaluating the received blood analyte data, the evaluating comprising calculating at least one parameter; based at least on the at least one parameter, configuring at least one user interface alert function, the configuring the at least one alert function comprising establishing a first buffer range of blood analyte levels above a normal range, and a second buffer range of blood analyte levels below the normal range; causing only a first type of alert to be provided to the user when the blood analyte level indicated by the at least one parameter falls within the first or second buffer ranges; and causing only a second type of alert to be provided to the user when the blood analyte level indicated by the at least one parameter falls outside of the first and second buffer ranges and the normal range; wherein the causing only a first type of alert to be provided to the user when the blood analyte level indicated by the at least one parameter falls within the first or second buffer ranges, and only a second type of alert to be provided to the user when the blood analyte level indicated by the at least one parameter falls outside of the first and second buffer ranges and the normal range, reduces the frequency of the second type of alerts provided to the user via the at least one user interface over that provided without use of the first and second buffer ranges.

In yet another aspect, a method of monitoring a blood analyte level of a living being using a blood analyte sensing apparatus comprising an implanted sensor apparatus having at least a prescribed stability of blood analyte level measurement over a prescribed period of time, and a receiver apparatus, is disclosed. In one embodiment, the method includes: receiving at the receiver apparatus blood analyte data from the implanted sensor apparatus, the blood analyte data associated with at least one first time reference; determining a time reference associated with a last calibration of the blood analyte sensing apparatus; determining a difference between the at least one first time reference and the time reference associated with the last calibration; evaluating the received blood analyte data, the evaluating comprising: calculating at least (i) a blood analyte level and (ii) a rate-of-change (ROC) of the blood analyte level; and configuring at least one user interface alert function, the configuring the at least one alert function comprising establishing a first buffer range of blood analyte levels above a normal range, and a second buffer range of blood analyte levels below the normal range, a magnitude of at least one of the first range and second range being selected based at least on (i) the difference, (ii) the calculated blood analyte level, and (iii) the calculated ROC.

Other features and advantages of the present disclosure will immediately be recognized by persons of ordinary skill in the art with reference to the attached drawings and detailed description of exemplary embodiments as given below.

### BRIEF DESCRIPTION OF THE DRAWINGS

FIG. 1 is a logical block diagram illustrating a typical prior art transcutaneous blood analyte monitoring system with external receiver.

FIGS. 1A and 1B show exemplary user interface (UI) displays associated with the exemplary prior art system of FIG. 1.

FIG. 2 is a front perspective view of one exemplary embodiment of a fully implantable biocompatible sensor apparatus useful with various aspects of the present disclosure.

FIGS. 2A-2C are top, bottom, and side elevation views, respectively, of the exemplary sensor apparatus of FIG. 2.

FIG. 3 is a logical block diagram illustrating one embodiment of a system architecture for, inter alia, monitoring blood analyte levels within a user, according to the present disclosure.

FIG. 3A is a logical block diagram illustrating another embodiment of a system architecture for, inter alia, monitoring blood analyte levels within a user, according to the present disclosure.

FIG. 3B is a logical block diagram illustrating yet another embodiment of a system architecture for, inter alia, monitoring blood analyte levels within a user, according to the present disclosure.

FIG. 3C is a functional block diagram illustrating an exemplary implantable sensor apparatus and local receiver apparatus according to one embodiment of the present disclosure.

FIG. 4 is a functional block diagram illustrating an exemplary embodiment of the local receiver apparatus of FIG. 3.

FIG. 4A is a functional block diagram illustrating an exemplary embodiment of the dedicated receiver and processor apparatus of FIG. 3A.

FIG. 4B is a functional block diagram illustrating another exemplary embodiment of the local receiver apparatus of FIG. 3A, wherein a biocompatible (e.g., implanted) output receiver is used in conjunction therewith.

FIG. 5 is a logical flow diagram illustrating one exemplary embodiment of a method of operating a receiving device for blood analyte measurement according to the present disclosure.

FIG. 5A is a logical flow diagram illustrating one exemplary implementation of the sensor data processing and output according to the method of FIG. 5.

FIG. 6A is a front perspective view of one exemplary embodiment of a blood analyte receiver and processor apparatus including a graphical display device for, inter alia, display of a user interface (UI).

FIG. 6B is a front perspective view of one exemplary embodiment of a local receiver apparatus in wireless communication with a parent platform apparatus including a graphical display for display of a user interface (UI).

FIG. 7 is a logical flow diagram illustrating one exemplary embodiment of a method of operating a UI for provision of blood analyte measurement data to a user, according to the present disclosure.

FIG. 7A illustrates several exemplary UI display configurations for display of current blood analyte level according to the present disclosure.

FIGS. 7B-1 and 7B-2 are logical tree diagrams of exemplary logic associated with user-specified parameters for user prompting and display via the UI.

FIG. 7C illustrates exemplary UI displays for receipt of user-specified Ideal Range (IR) and daytime Buffer Zone Ranges (BZRs).

FIG. 7D-1 illustrates exemplary UI displays for receipt of user-specified nighttime Buffer Zone Ranges (BZRs) and a nighttime time period.

FIG. 7D-2 illustrates an exemplary embodiment of an integrated daytime and nighttime UI display including common display of all relevant values.

FIG. 7E is a logical flow diagram illustrating one exemplary implementation of the provision of alert notifications according to the method of FIG. 7.

FIG. 7F illustrates exemplary UI displays for notification of required user action/attention and receipt of user input.

FIG. 7G illustrates exemplary UI displays for receipt of user action input and a user action log.

FIG. 7H is a logical flow diagram illustrating one exemplary implementation of blood analyte level data analysis according to the method of FIG. 7.

FIG. 7I illustrates exemplary UI display for trend line graphical analysis of blood analyte level data and user input data.

FIG. 7J illustrates exemplary UI displays for bar-type graphical analysis of blood analyte level data.

FIG. 7K illustrates an exemplary UI display for non-graphical analysis of blood analyte level data.

FIG. 7L illustrates an exemplary UI display of a toggle menu for receiving selections of a desired blood analyte level output for display on the UI.

FIG. 7M is a logical flow diagram illustrating one exemplary implementation of data error identification and/or mitigation according to the method of FIG. 7.

FIG. 7N is a graph illustrating exemplary selectable sound alert profile modes for a local receiver apparatus, parent platform, and/or a dedicated receiver and processor apparatus according to the disclosure.

FIGS. 8 and 8A are logical flow diagrams illustrating exemplary embodiments of methods for utilizing context data in conjunction with a monitoring and/or alert regimes associated with blood analyte sensing systems.

FIG. 8B is a functional block diagram illustrating an exemplary embodiment of the local receiver apparatus of FIG. 3, configured to implement the methodologies of FIGS. 8 and 8A and having indigenous sensors and network connectivity to a networked computerized personal assistant application.

FIGS. 9A-9D are schematic diagrams illustrating various exemplary use-cases of the blood analyte detection system and UI according to the present disclosure.

All Figures© Copyright 2016-2017 GlySens Incorporated. All rights reserved.

### DETAILED DESCRIPTION

Reference is now made to the drawings, wherein like numerals refer to like parts throughout.

### Overview

One aspect of the present disclosure leverages Assignee's recognition that many of the above-described disabilities of the prior art for providing data or other information of value via a user interface (UI) associated with a blood analyte detection system (e.g.,, an implanted analyte sensor in communication with a receiver, etc.) can be mitigated or even completely eliminated via enabling personalized “context-aware” and dynamic detection of blood analyte level, further enabled by receipt of user input and customized notifications to the user via the UI.

Accordingly, the present disclosure makes use in one exemplary embodiment of an improved UI and associated processing logic (as well as functionality for the UI in combination with a receiving apparatus and/or other device in data communication with the blood analyte detection system), so as to overcome the limitations of the prior art such as e.g.: (i) provision of alerts or notifications only at times when the user's blood analyte level is at a level which could be hazardous to the user's health (i.e., when the level is in fact a “problem”); (ii) overly frequent and intrusive alerting; and/or (iii) limited mechanisms for analysis of data (and hence enabling extraction of useful information for the user regarding the data such as observed patterns).

The methods and apparatus of the present disclosure also, in one embodiment, leverage the only recently-available user “freedom” provided by implantable sensors; i.e., capability for implanted blood analyte sensing devices such as the oxygen-based blood glucose sensing device manufactured by the Assignee hereof, to remain implanted for extended periods, provide highly stable data, and require only very intermittent (e.g., once per week) calibration via e.g., external fingerstick device. Such advantages of extended implantation, stability, and only intermittent external calibration could easily be frustrated if coupled with a user interface, monitoring and/or alert scheme which do not take such capabilities into account or use them to their full advantage.

In one implementation, the aforementioned implantable sensor (e.g., an oxygen-based sensor for detection of blood glucose (BG) level) is used in conjunction with either a local receiver apparatus in data communication with a parent platform (e.g., a user's mobile device), or a dedicated receiver and processor apparatus (or both). Each of the foregoing devices includes a UI for providing notifications to the user according to the aforementioned contextual awareness and received user inputs for personalized analyte level detection, notification, and analysis. Hence, the user experience and quality of life are substantially improved through personalization of the blood analyte detection system via the UI and associated logic disclosed herein.

Specifically, in one variant, the aforementioned UI and associated logic are configured to receive and store user-specified parameters for operation of the blood analyte detection system including an Ideal Range (IR), and high and low Buffer Zone Ranges (BZRs) for blood analyte level. This approach enables definition of a Priority Range (PR); i.e., a blood analyte level that lies outside of the high and low BZRs. The BZRs enable provision of pre-alert notifications (i.e., progressive “soft” alert notifications) via the UI as the blood analyte level approaches a level of greater concern within the PR. Advantageously, the BZRs can be selectively turned on and off, and be made context-dependent (e.g., time-of-day dependent (e.g., nighttime BZRs and daytime BZRs), day of the week-dependent, activity-dependent (e.g., when engaging in activity or behavior that is anticipated to create more rapid and/or greater magnitude variations in blood glucose level than other times). The alert regime or mode can also be customized by the user; e.g., certain contexts can be associated with a milder alert (as compared to say a PR-associated alert). In another variant, the user's context is actively sensed by the receiver/parent apparatus (whether using indigenous means, or those of another platform). For example, an indigenous accelerometer disposed in the local receiver apparatus (e.g., small-profile wristband or fob) can be utilized to detect motion or activity level of the user; with only periodic detected motion, the user can be presumed to be asleep or resting, and accordingly the prevailing monitoring and/or alert regime adjusted accordingly. Conversely, very frequent detection of high-magnitude motion may be indicative of the user engaging in strenuous activity such as running or cross-fit, thereby dictating a different monitoring/alert regime.

User “device” context may also be utilized by the exemplary embodiments of the monitoring system to select/configure monitoring and/or alert regimes to be implemented.

For example, in one implementation, the logic of the system is configured to determine which UI-enabled devices are in active use by the user, and accordingly structure the monitoring/alert regime(s) based thereon, so as make most effective use of the particular UI capabilities of those active devices. In yet other variants, the UI and associated logic are configured to (i) selectively display various analyses of current blood analyte level data and/or historical blood analyte level data; (ii) prompt, receive, and store user action input data; and/or (iii) identify and mitigate data errors.

Methods of use of the aforementioned UI and blood analyte detection system are also disclosed herein.

### Detailed Description of Exemplary Embodiments

Exemplary embodiments of the present disclosure are now described in detail. While these embodiments are primarily discussed in the context of a fully implantable glucose sensor, such as those exemplary embodiments described herein, and/or those set forth in U.S. Patent Application Publication No. 2013/0197332 filed Jul. 26, 2012 entitled “Tissue Implantable Sensor With Hermetically Sealed Housing;” U.S. Pat. No. 7,894,870 to Lucisano et al. issued Feb. 22, 2011 and entitled “Hermetic Implantable Sensor;” U.S. Patent Application Publication No. 2011/0137142 to Lucisano et al. published Jun. 9, 2011 and entitled “Hermetic Implantable Sensor;” U.S. Pat. No. 8,763,245 to Lucisano et al. issued Jul. 1, 2014 and entitled “Hermetic Feedthrough Assembly for Ceramic Body;” U.S. Patent Application Publication No. 2014/0309510 to Lucisano et al. published Oct. 16, 2014 and entitled “Hermetic Feedthrough Assembly for Ceramic Body;” U.S. Pat. No. 7,248,912 to Gough et al. issued Jul. 24, 2007 and entitled “Tissue Implantable Sensors for Measurement of Blood Solutes;” and U.S. Pat. No. 7,871,456 to Gough et al. issued Jan. 18, 2011 and entitled “Membranes with Controlled Permeability to Polar and Apolar Molecules in Solution and Methods of Making Same;” and U.S. Patent Application Publication No. 2013/0197332 to Lucisano et al. published Aug. 1, 2013 and entitled “Tissue Implantable Sensor with Hermetically Sealed Housing;” PCT Patent Application Publication No. 2013/016573 to Lucisano et al. published Jan. 31, 2013 and entitled “Tissue Implantable Sensor with Hermetically Sealed Housing,” each of the foregoing incorporated herein by reference in its entirety, as well as those of U.S. patent application Ser. Nos. 13/559,475, 14/982,346, 15/170,571, and 15/197,104, 15/359,406, and 15/368,436 previously incorporated herein, it will be recognized by those of ordinary skill that the present disclosure is not so limited. In fact, the various aspects of the disclosure are useful with, inter alia, other types of implantable sensors and/or electronic devices.

Further, while the following embodiments describe specific implementations of e.g., biocompatible oxygen-based multi-sensor element devices for measurement of glucose, having specific configurations, protocols, locations, and orientations for implantation (e.g., proximate the waistline on a human abdomen with the sensor array disposed proximate to fascial tissue; see e.g., U.S. patent application Ser. No. 14/982,346 filed Dec, 29, 2015 and entitled “Implantable Sensor Apparatus and Methods” previously incorporated herein), those of ordinary skill in the related arts will readily appreciate that such descriptions are purely illustrative, and in fact the methods and apparatus described herein can be used consistent with, and without limitation: (i) in living beings other than humans; (ii) other types or configurations of sensors (e.g., other types, enzymes, and/or theories of operation of glucose sensors, sensors other than glucose sensors, such as e.g., sensors for other analytes such as urea, lactate); (iii) other implantation locations and/or techniques (including without limitation transcutaneous or non-implanted devices as applicable); and/or (iv) devices intended to deliver substances to the body (e.g. implanted drug pumps, drug-eluting solid materials, and encapsulated cell-based implants, etc.); and/or other devices (e.g., non-sensors and non-substance delivery devices).

As used herein, the term “analyte” refers without limitation to a substance or chemical species that is of interest in an analytical procedure. In general, the analyte itself may or may not be directly measurable, in cases where it is not, a measurement of the analyte (e.g., glucose) can be derived through measurement of chemical constituents, components, or reaction byproducts associated with the analyte (e.g., hydrogen peroxide, oxygen, free electrons, etc.).

As used herein, the terms “detector” and “sensor” refer without limitation to a device having one or more elements (e.g., detector element, sensor element, sensing elements, etc.) that generate, or can be made to generate, a signal indicative of a measured parameter, such as the concentration of an analyte (e.g., glucose) or its associated chemical constituents and/or byproducts (e.g., hydrogen peroxide, oxygen, free electrons, etc.). Such a device may be based on electrochemical, electrical, optical, mechanical, thermal, or other principles as generally known in the art. Such a device may consist of one or more components, including for example, one, two, three, or four electrodes, and may further incorporate immobilized enzymes or other biological or physical components, such as membranes, to provide or enhance sensitivity or specificity for the analyte.

As used herein, the terms “orient,” “orientation,” and “position” refer, without limitation, to any spatial disposition of a device and/or any of its components relative to another object or being, and in no way connote an absolute frame of reference.

As used herein, the terms “top,” “bottom,” “side,” “up,” “down,” and the like merely connote, without limitation, a relative position or geometry of one component to another, and in no way connote an absolute frame of reference or any required orientation. For example, a “top” portion of a component may actually reside below a “bottom” portion when the component is mounted to another device (e.g., host sensor).

As used herein the term “parent platform” refers without limitation to any device, group of devices, and/or processes with which a client or peer device (including for example the various embodiments of local receiver described here) may logically and/or physically communicate to transfer or exchange data. Examples of parent platforms can include, without limitation, smartphones, tablet computers, laptops, smart watches, personal computers/desktops, servers (local or remote), gateways, dedicated or proprietary analyte receiver devices, medical diagnostic equipment, and even other local receivers acting in a peer-to-peer or dualistic (e.g., master/slave) modality.

As used herein, the term “application” (or “app”) refers generally and without limitation to a unit of executable software that implements a certain functionality or theme. The themes of applications vary broadly across any number of disciplines and functions (such as on-demand content management, e-commerce transactions, brokerage transactions, home entertainment, calculator etc.), and one application may have more than one theme. The unit of executable software generally runs in a predetermined environment; for example, the Java® environment.

As used herein, the term “computer program” or “software” is meant to include any sequence or human or machine cognizable steps which perform a function. Such program may be rendered in virtually any programming language or environment including, for example, C/C++, Fortran, COBOL, PASCAL, assembly language, markup languages (e.g., HTML, SGML, XML, VoXML), and the like, as well as object-oriented environments such as the Common Object Request Broker Architecture (CORBA), Java® (including J2ME, Java Beans, etc.) and the like.

As used herein, the terms “Internet” and “internet” are used interchangeably to refer to inter-networks including, without limitation, the Internet. Other common examples include but are not limited to: a network of external servers, “cloud” entities (such as memory or storage not local to a device, storage generally accessible at any time via a network connection, or cloud-based or distributed processing or other services), service nodes, access points, controller devices, client devices, etc.

As used herein, the term “memory” includes any type of integrated circuit or other storage device adapted for storing digital data including, without limitation, ROM, PROM, EEPROM, DRAM, SDRAM, DDR/2 SDRAM, EDO/FPMS, RLDRAM, SRAM, “flash” memory (e.g., NAND/NOR), 3D memory, and PSRAM.

As used herein, the terms “microprocessor” and “processor” or “digital processor” are meant generally to include all types of digital processing devices including, without limitation, digital signal processors (DSPs), reduced instruction set computers (RISC), general-purpose (CISC) processors, microprocessors, gate arrays (e.g., FPGAs), PLDs, state machines, reconfigurable computer fabrics (RCFs), array processors, secure microprocessors, and application-specific integrated circuits (ASICs). Such digital processors may be contained on a single unitary integrated circuit (IC) die, or distributed across multiple components.

As used herein, the term “network” refers generally to any type of telecommunications or data network including, without limitation, hybrid fiber coax (HFC) networks, satellite networks, telco networks, and data networks (including MANs, WANs, LANs, WLANs, internets, and intranets), cellular networks, as well as so-called “mesh” networks and “IoTs” (Internet(s) of Things). Such networks or portions thereof may utilize any one or more different topologies (e.g., ring, bus, star, loop, etc.), transmission media (e.g., wired/RF cable, RF wireless, millimeter wave, optical, etc.) and/or communications or networking protocols.

As used herein, the term “interface” refers to any signal or data interface with a component or network including, without limitation, those of the FireWire (e.g., FW400, FW800, etc.), USB (e.g., USB 2.0, 3.0. OTG), Ethernet (e.g., 10/100, 10/100/1000 (Gigabit Ethernet), 10-Gig-E, etc.), MoCA, LTE/LTE-A, Wi-Fi (802.11), WiMAX (802.16), Z-wave, PAN (e.g., 802.15)/Zigbee, Bluetooth, or power line carrier (PLC) families.

As used herein, the term “storage” refers to without limitation computer hard drives, memory, RAID devices or arrays, optical media (e.g., CD-ROMs, Laserdiscs, Blu-Ray, etc.), solid state devices (SSDs), flash drives, cloud-hosted storage, or network attached storage (NAS), or any other devices or media capable of storing data or other information.

As used herein, the term “Wi-Fi” refers to, without limitation and as applicable, any of the variants of IEEE-Std. 802.11 or related standards including 802.11 a/b/g/n/s/v/ac or 802.11-2012/2013, as well as Wi-Fi Direct (including inter alia, the “Wi-Fi Peer-to-Peer (P2P) Specification”, incorporated herein by reference in its entirety).

As used herein, the term “wireless” means any wireless signal, data, communication, or other interface including without limitation Wi-Fi, Bluetooth, 3G (3GPP/3GPP2), HSDPA/HSUPA, TDMA, CDMA (e.g., IS-95A, WCDMA, etc.), FHSS, DSSS, GSM, PAN/802.15, WiMAX (802.16), 802.20, Zigbee®, Z-wave, narrowband/FDMA, OFDM, PCS/DCS, LTE/LTE-A, analog cellular, CDPD, satellite systems, millimeter wave or microwave systems, acoustic, and infrared (i.e., IrDA).

### Exemplary Implantable Sensor

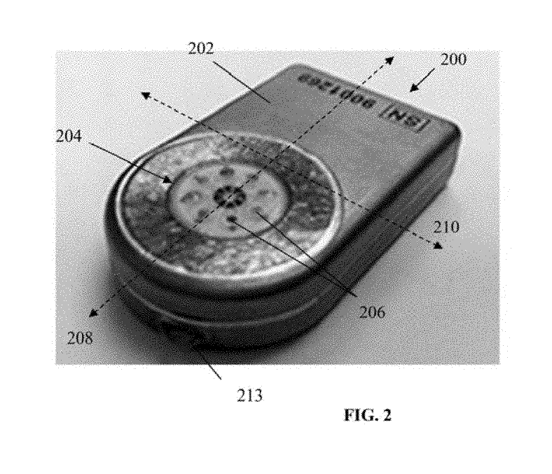

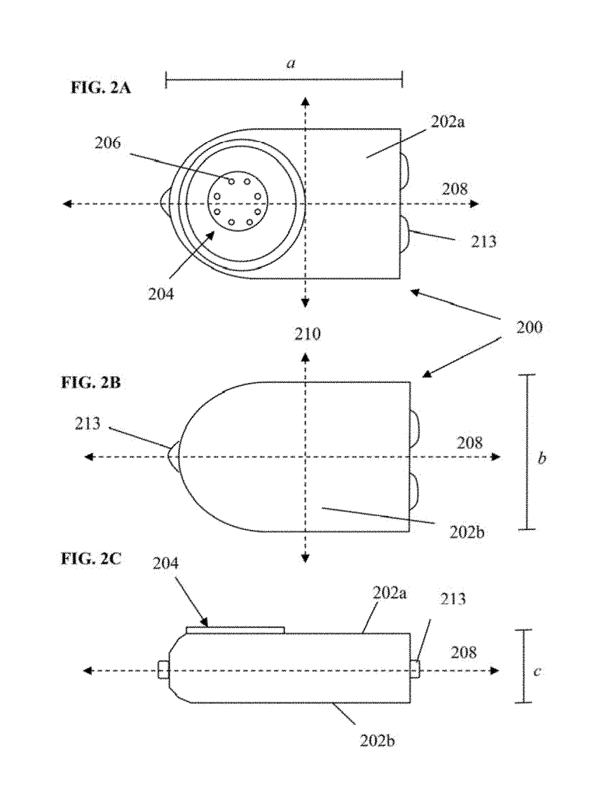

Referring now to FIGS. 2-2C, one exemplary embodiment of a sensor apparatus useful with various aspects of the present disclosure is shown and described.

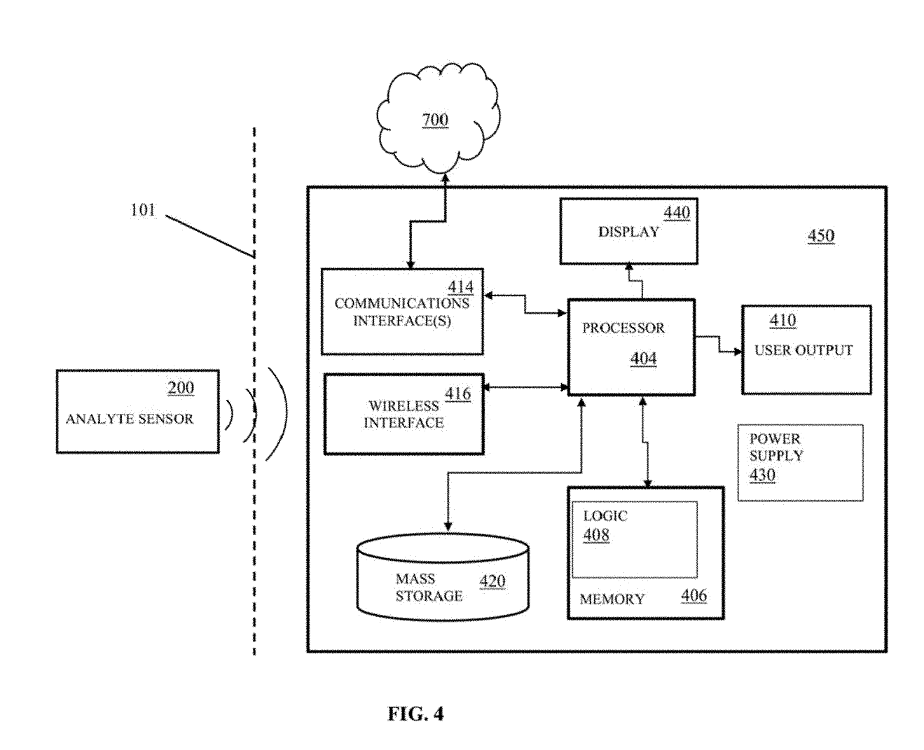

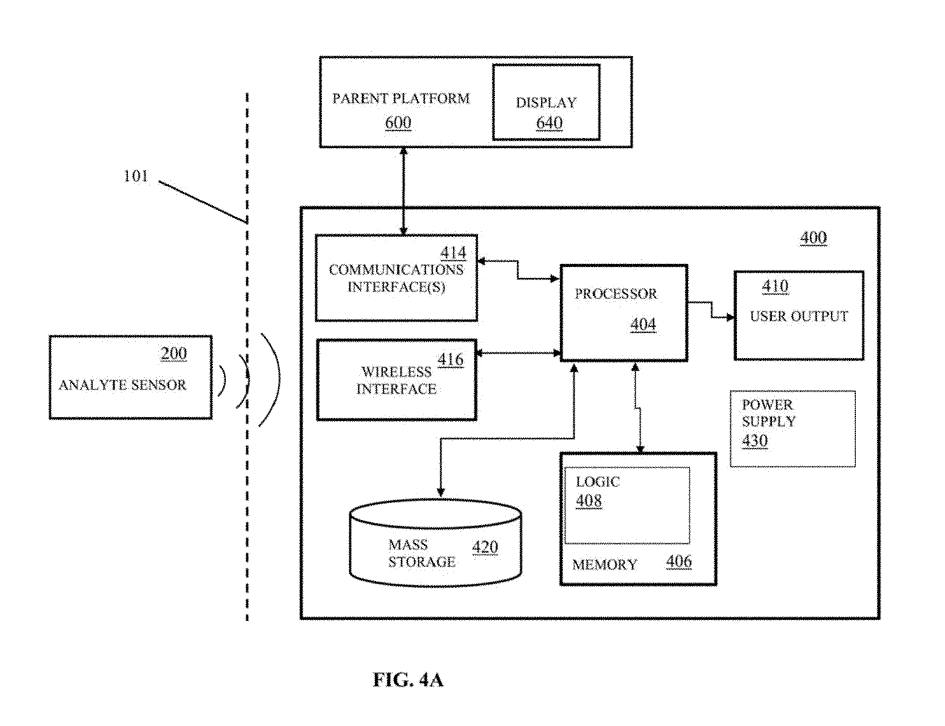

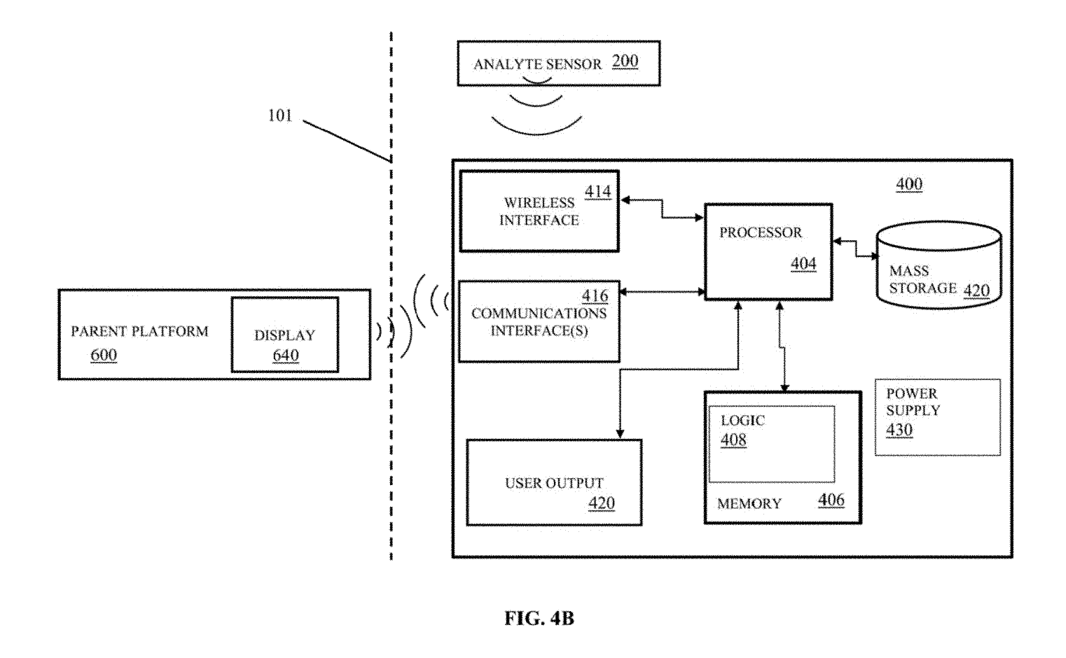

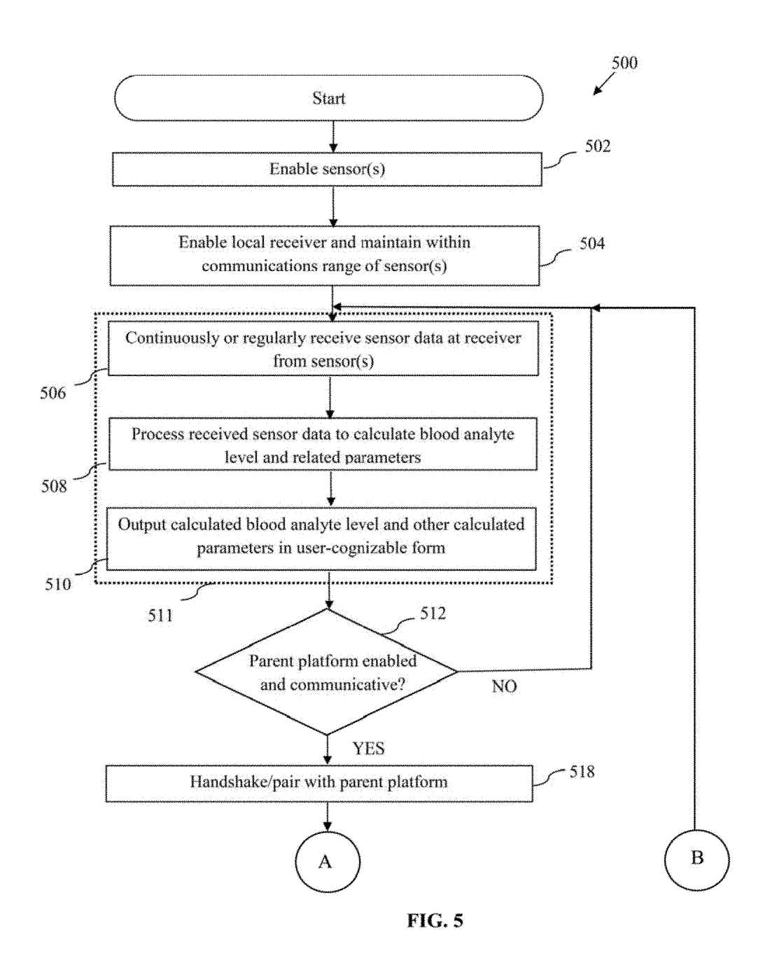

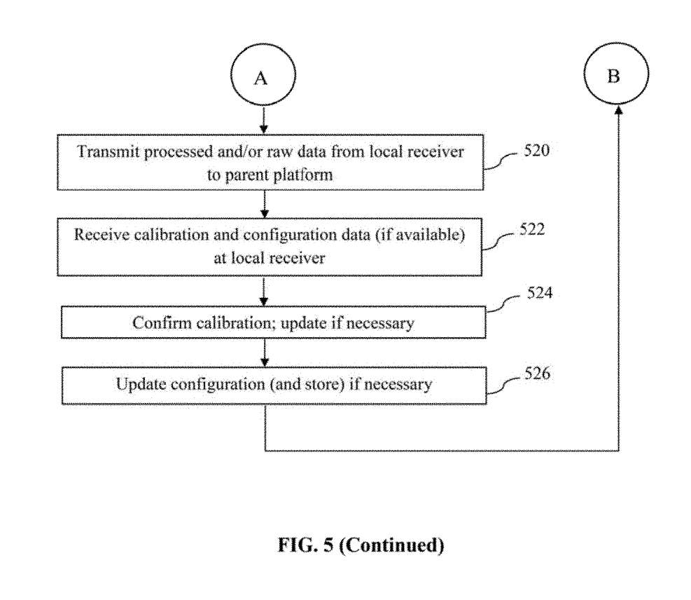

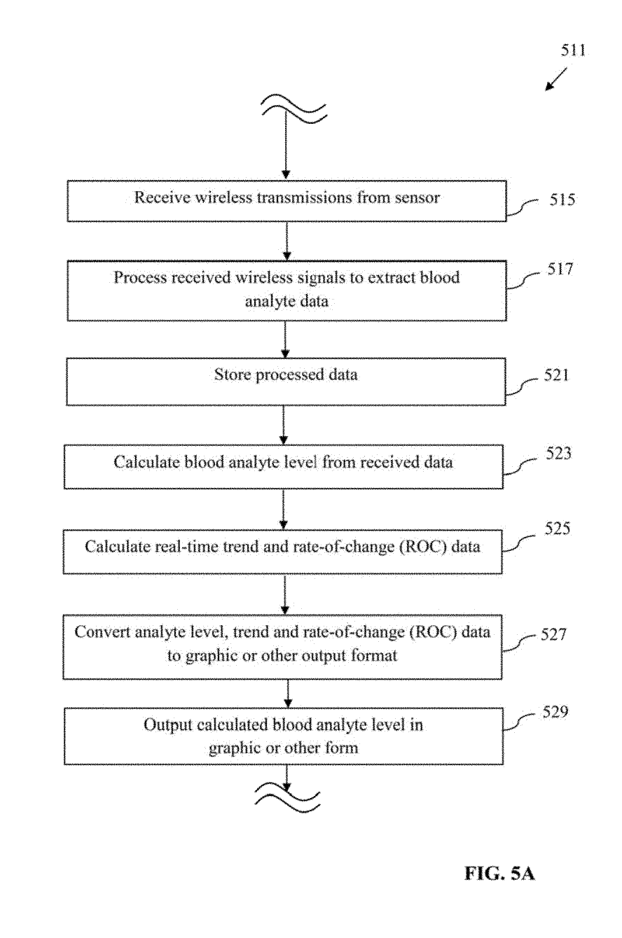

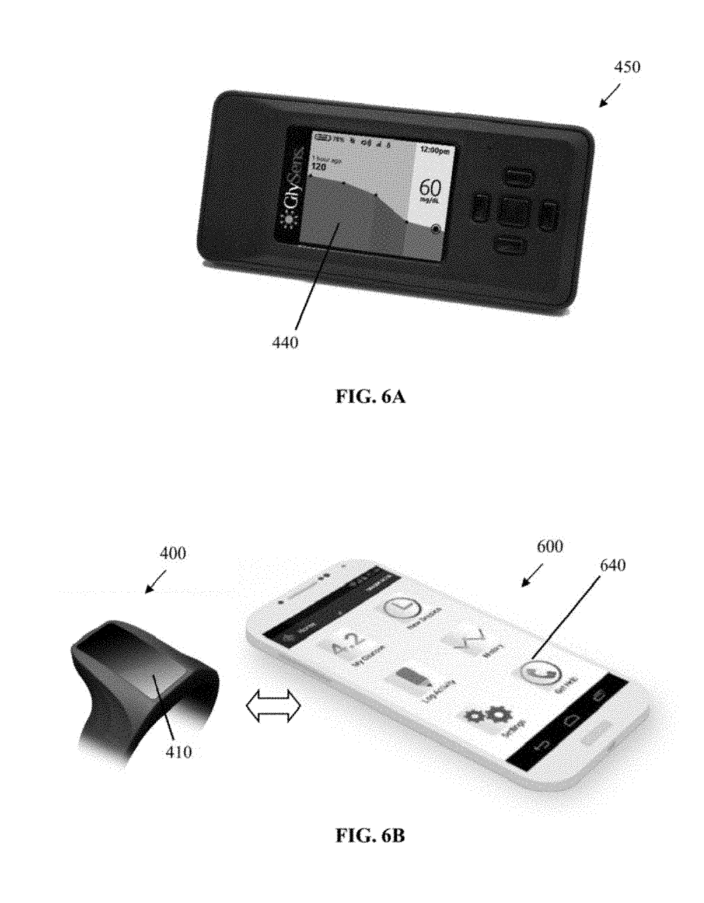

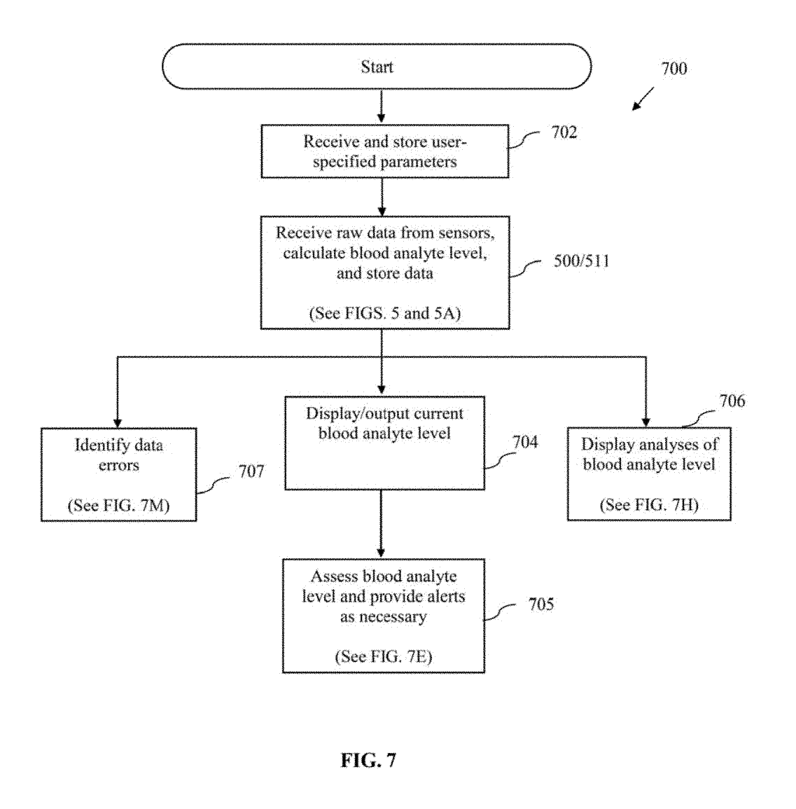

As shown in FIGS. 2-2C, the exemplary sensor apparatus 200 comprises a somewhat planar housing structure 202 with a sensing region 204 disposed on one side thereof (i.e., a top face 202a). As described in greater detail below with respect to FIGS. 4-5, the exemplary substantially planar shape of the housing 202 provides mechanical stability for the sensor apparatus 200 after implantation, thereby helping to preserve the orientation of the apparatus 200 and mitigate any tissue response induced by movement of the apparatus while implanted. Notwithstanding, the present disclosure contemplates sensor apparatus of shapes and/or sizes other than that of the exemplary apparatus 200.

The sensor apparatus of FIGS. 2-2C further includes a plurality of individual sensor elements 206 with their active surfaces disposed substantially within the sensing region 204 on the top face 202a of the apparatus housing. In the exemplary embodiment (i.e., an oxygen-based glucose sensor), the eight (8) sensing elements 206 are grouped into four pairs, one element of each pair an active or “primary” sensor with enzyme matrix, and the other a reference or “secondary” (oxygen) sensor. Exemplary implementations of the sensing elements and their supporting circuitry and components are described in, inter alia, U.S. Pat. No. 7,248,912, previously incorporated herein. It will be appreciated, however, that the type and operation of the sensor apparatus may vary; i.e., other types of sensor elements/sensor apparatus, configurations, and signal processing techniques thereof may be used consistent with the various aspects of the present disclosure, including, for example, signal processing techniques based on various combinations of signals from individual elements in the otherwise spatially-defined sensing elements pairs.

The illustrated embodiment of FIGS. 2-2C includes a sensing region 204 which facilitates some degree of “interlock” of the surrounding tissue (and any subsequent tissue response generated by the host) so as to ensure direct and sustained contact between the sensing region 204 and the blood vessels of the surrounding tissue during the entire term of implantation (as well as advantageously maintaining contact between the sensing region 204 and the same tissue; i.e., without significant relative motion between the two).

The sensor apparatus 200 also includes in the exemplary embodiment a wireless radio frequency transmitter (or transceiver, depending if signals are intended to be received by the apparatus), not shown. As described in the aforementioned documents incorporated herein, the transmitter/transceiver may be configured to transmit modulated radio frequency signals to an external receiver/transceiver, such as a dedicated receiver device, or alternatively a properly equipped consumer electronic device such as a smartphone or tablet computer. Moreover, the sensor apparatus 200 may be configured to transmit signals to (whether in conjunction with the aforementioned external receiver, or in the alternative) an at least partly implanted or in vivo receiving device, such as an implanted pump or other medication or substance delivery system (e.g., an insulin pump or dispensing apparatus), embedded “logging” device, or other. It is also appreciated that other forms of wireless communication may be used for such applications, including for example inductive (electromagnetic induction) based systems, or even those based on capacitance or electric fields, or even optical (e.g., infrared) systems where a sufficiently clear path of transmission and reception exists, such as two devices in immediately adjacent disposition, or even ultrasonic systems where the two devices are sufficiently close and connected by sound-conductive media such as body tissues or fluids, or a purposely implanted component.

The sensor apparatus of FIGS. 2-2C also includes a plurality (three in this instance) of tabs or anchor apparatus 213 disposed substantially peripheral on the apparatus housing. These anchor apparatus provide the implanting surgeon with the opportunity to anchor the apparatus to the anatomy of the living subject, so as to frustrate translation and/or rotation of the sensor apparatus 200 within the subject immediately after implantation but before any tissue response (e.g., FBR) of the subject has a chance to immobilize (such as via interlock with the sensing region of the apparatus. See e.g., U.S. patent application Ser. No. 14/982,346 filed Dec. 29, 2015 and entitled “Implantable Sensor Apparatus and Methods” previously incorporated herein, for additional details and considerations regarding the aforementioned anchor apparatus 213 (which may include, for example features to receive sutures (dissolvable or otherwise), tissue ingrowth structures, and/or the like).

Moreover, another exemplary embodiment of the sensor apparatus 200 described herein may include either or both of: (i) multiple detector elements with respective “staggered” ranges/rates of detection operating in parallel, and/or (ii) multiple detector elements with respective “staggered” ranges/rates of detection that are selectively switched on/off in response to, e.g., the analyte concentration reaching a prescribed upper or lower threshold, as described in the foregoing patent application Ser. No. 15/170,571 previously incorporated herein.

The present disclosure further contemplates that such thresholds or bounds: (i) can be selected independent of one another; and/or (ii) can be set dynamically while the apparatus 300 is implanted. For example, in one scenario, operational detector elements are continuously or periodically monitored to confirm accuracy, and/or detect any degradation of performance (e.g., due to equipment degradation, progressive FBR affecting that detector element, etc.); when such degradation is detected, affecting say a lower limit of analyte concentration that can be detected, that particular detector element can have its lower threshold adjusted upward, such that handoff to another element capable of more accurately monitoring concentrations in that range. Note that these thresholds or bounds are to be distinguished from those associated with the user interface (UI) described subsequently herein, the latter being independent of the data source/capability/configuration associated with the sensor detector elements.

It will be appreciated that the relatively smaller dimensions of the sensor apparatus (as compared to many conventional implant dimensions)—on the order of 40 mm in length (dimension “a” on FIGS. 2A-2C) by 25 mm in width (dimension “b” on FIGS. 2A-2C) by 10 mm in height (dimension “c” on FIGS. 2A-2C)—may reduce the extent of injury (e.g., reduced size of incision, reduced tissue disturbance/removal, etc.) and/or the surface area available for blood/tissue and sensor material interaction, which may in turn reduce intensity and duration of the host wound healing response. It is also envisaged that as circuit integration is increased, and component sizes (e.g., Lithium or other batteries) decrease, and further improvements are made, the sensor may increasingly be appreciably miniaturized, thereby further leveraging this factor.

System Architecture—

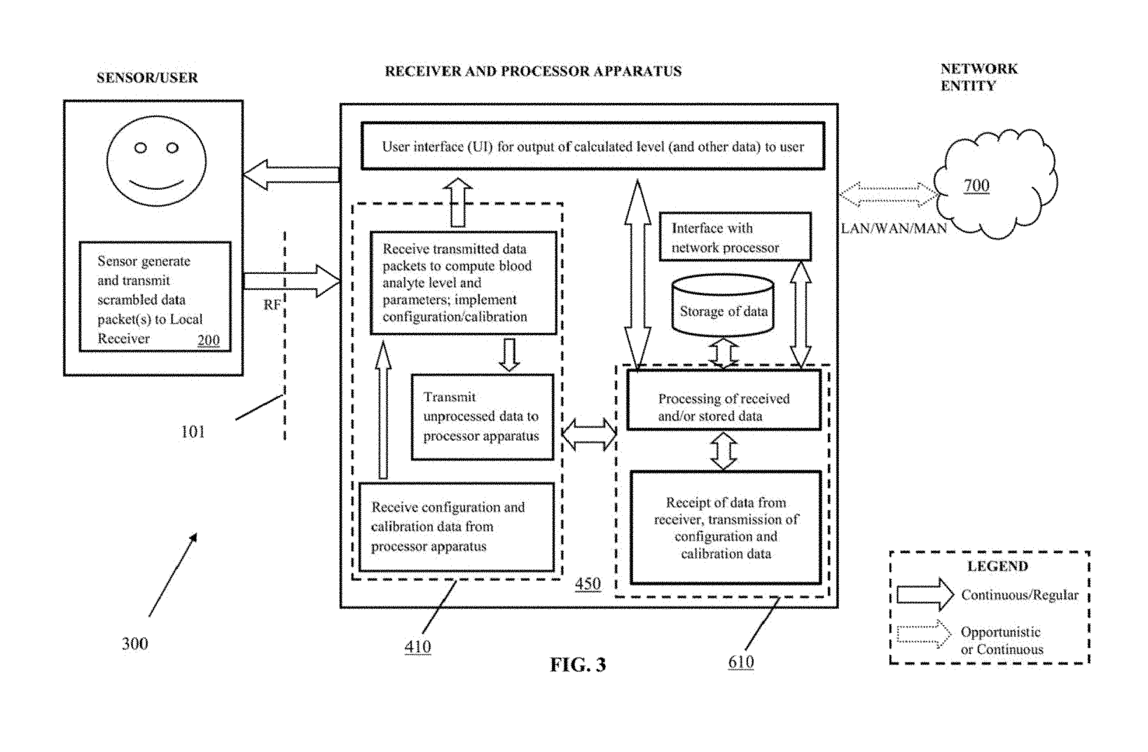

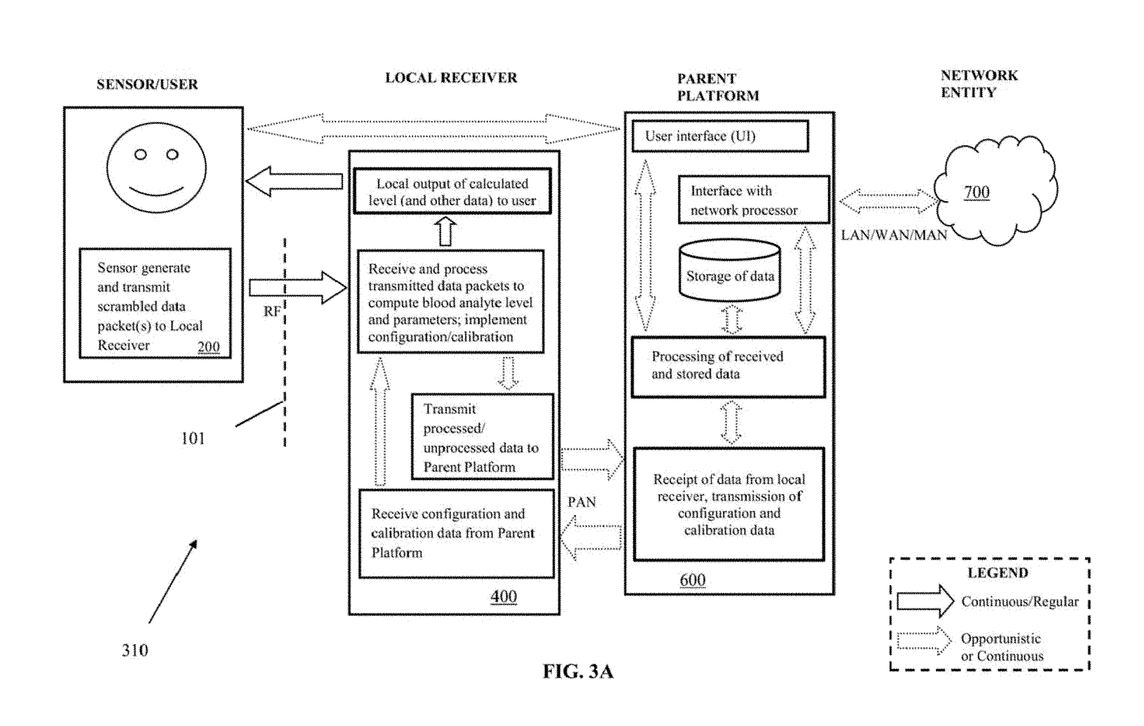

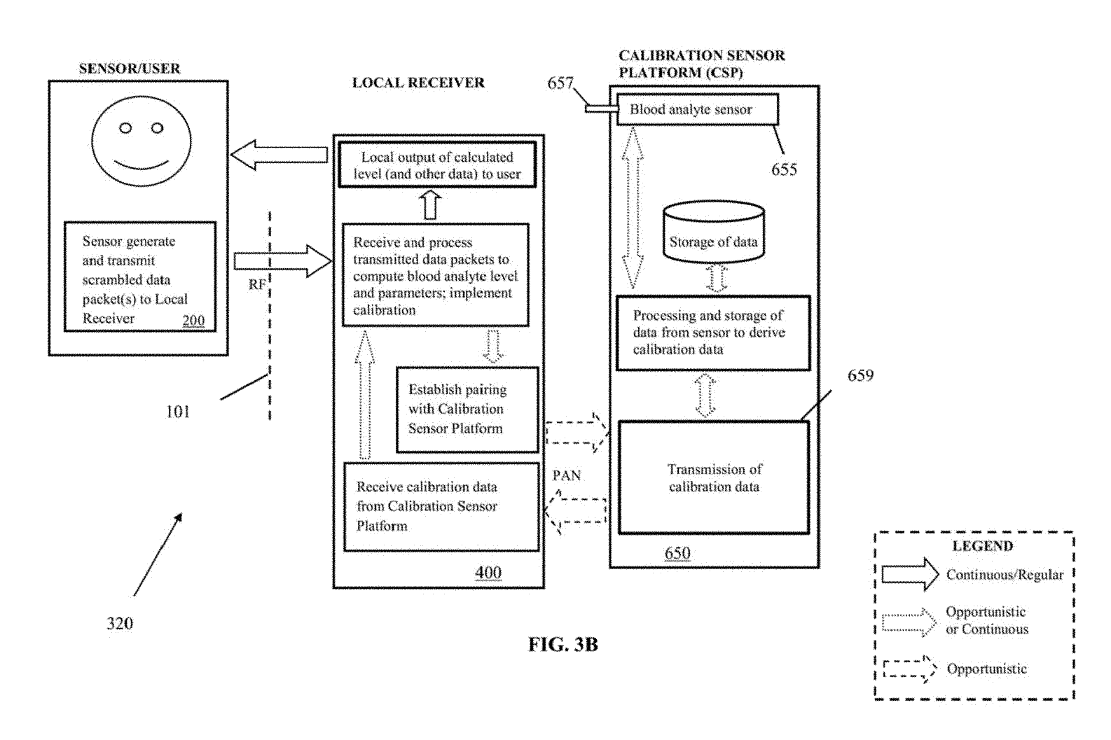

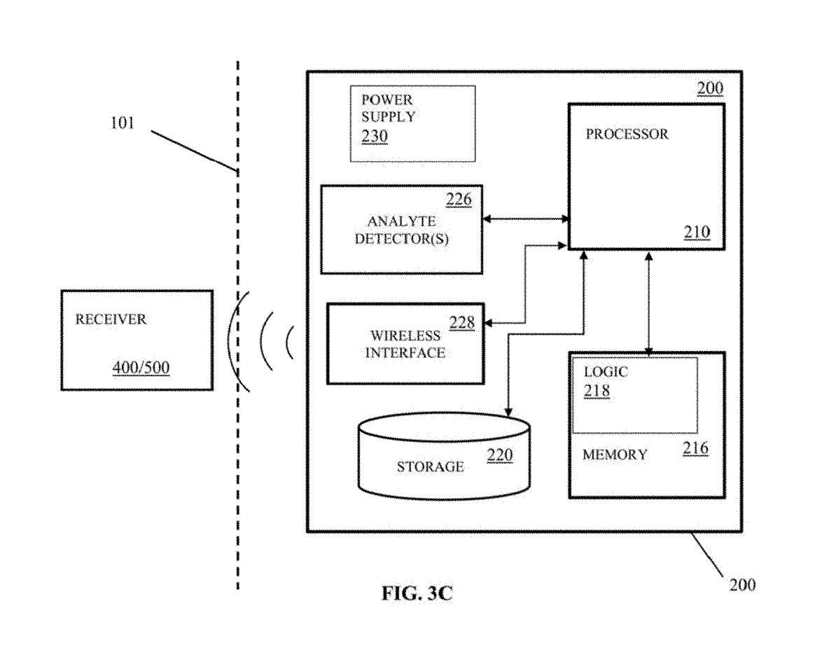

Referring now to FIG. 3, one embodiment of a system architecture for, inter alia, monitoring blood analyte levels within a user, according to the present disclosure is described in detail. As depicted in FIG. 3, architecture 300 comprises a sensor apparatus 200 (e.g., that of FIG. 2 discussed above, or yet other types of device) associated with a user, a receiver and processor apparatus 450 and a network entity 700. The sensor apparatus 200 in this embodiment communicates with the receiver and processor apparatus 450 via a wireless interface (described in detail below) through the user's tissue boundary 101. The receiver and processor apparatus 450 may also, if desired, communicate with one or more network entities 700 via a LAN/WLAN, MAN, or other topology, such as for “cloud” data storage, analysis, convenience of access at other locations/synchronization with other user platforms, etc.

As shown in FIG. 3A, another system architecture 310 comprises a sensor apparatus 200 (e.g., that of FIG. 2 discussed above, or yet other types of device) associated with a user, a local receiver 400, a parent platform 600, and a network entity 700. The sensor apparatus 200 in this embodiment communicates with the local receiver 400 via a wireless interface (described in detail below) through the user's tissue boundary 101. The local receiver 400 communicates (e.g., wirelessly) with the one or more parent platform(s) 600 via a PAN (e.g., Bluetooth or similar) RF interface, as discussed in greater detail below. The parent platform 600 may also, if desired, communicate with one or more network entities 700 via a LAN/WLAN, MAN, or other topology, such as for “cloud” data storage, analysis, convenience of access at other locations/synchronization with other user platforms, etc.

In exemplary system architecture shown in FIG. 3A, the local receiver 400 is a lower profile and/or wearable local receiver apparatus (e.g., small profile wristband, fob, tooth or other implant, skin-adherent patch, ear “bud” or plug, a ring worn in the finger, etc.) as compared to the receiver and processor apparatus 450 shown in FIG. 3. The local receiver apparatus 400 can include a user alert mechanism and/or minimal user interface (UI) such as, e.g., a substantially flat and flexible LED (e.g., graphene-based), AMOLED, or OTFT (organic thin-film transistor) display device, haptic mechanism (e.g., a vibration mechanism), auditory mechanism (e.g., speakers), and/or other user-signaling capabilities and mechanisms (e.g., indicator lights). Various exemplary configurations for the local receiver 400 are shown and described in U.S. patent application Ser. No. 15/368,436 filed Dec. 2, 2016 previously incorporated herein.

As indicated in FIG. 3A, the communications between the sensor 200 and the local receiver 400 are generally continuous and reliable in nature (similar to communication between the sensor 200 and the receiver and processor apparatus 450 of

FIG. 3). In contrast, the communication between the local receiver 400 and the parent platform(s) can be either continuous or opportunistic (or any desired combination thereof between the various components of each) in nature. Such opportunistic communication can be a significant advantage of the architecture 300 over the prior art; i.e., the ability for the sensor 200 and a reduced form-factor local receiver 400 to communicate regularly to enable reliable and effectively constant monitoring and user awareness of their blood analyte (e.g., glucose) level, without being “tethered” to larger, bulkier, and perhaps activity-limiting parent devices, including for extended periods of time.

Specifically, in the illustrated architecture 310, the local receiver 400 acts as a reduced- or limited-functionality indicator and monitor for the user that reliably operates for comparatively extended periods of time without external input or calibration, thereby obviating the parent platform during those periods. As described later herein, the reduced form factor advantageously enables the user to further: (i) engage in activities which they could not otherwise engage in if “tethered” to the parent platform, and (ii) effortlessly keep the local receiver with them at all times, and obtain reliable blood analyte data and other useful information (e.g., trend, rate of change (ROC), and other sensor-data derived parameters), in a non-obtrusive (or even covert) manner.

In the example of system architecture 310, when communication between the parent platform 600 and the local receiver does occur, the exemplary architecture 310 enables two-way data transfer, including: (i) transfer of stored data extracted from the sensor wireless transmissions to the local receiver, to the parent platform for archiving, analysis, transfer to a network entity, etc.; (ii) transfer of sensor-specific identification data and/or local receiver-specific data between the local receiver and the parent platform; (iii) transfer of external calibration data (e.g., derived from an independent test method such as a fingerstick or blood glucose monitor and input either automatically or manually to the parent platform) from the parent to the local receiver; and (iv) transfer of local receiver configuration or other data (e.g., software/firmware updates, user-prescribed receiver settings for alerts, warning/buffer values, indication formats or parameters, historical blood analyte levels for the user, results of analysis by the parent 600 or network entity 700 of such data, diagnoses, security or data scrambling/encryption codes or keys, etc.) from the parent 600 to the local receiver 400.

FIG. 3B shows another embodiment of a system architecture for, inter alia, monitoring blood analyte levels within a user, according to the present disclosure. As shown in FIG. 3B, the architecture 320 comprises a sensor apparatus 200 associated with a user, a local receiver 400, and calibration sensor platform 650. As with the embodiment of FIG. 3A, the sensor apparatus 200 in this embodiment communicates with the local receiver 400 via a wireless interface through the user's tissue boundary 101. The local receiver 400 communicates (e.g., wirelessly) with one or more calibration sensor platform(s) or CSPs 650 via a PAN (e.g., Bluetooth or similar) RF interface, as discussed in greater detail below, or via IR (e.g., IrDA-compliant), optical or other short-range communication modality. As described in greater detail below, the CSP 650 in the illustrated embodiment comprises a calibration data source for the local receiver 400, which may stand in the place of the more fully-functioned parent platform 600 for at least provision of calibration data.

As indicated in FIG. 3B, the communications between the sensor 200 and the local receiver 400 are again generally continuous or regular in nature while the communication between the local receiver 400 and the CSP 650 is purposely opportunistic in nature.

When the opportunistic communication between the CSP 650 and the local receiver does occur, the exemplary architecture 310 enables at least one-way data transfer, including transfer of external calibration data (e.g., derived from an independent test method such as the “fingerstick” or other form of blood analyte sensor 655 of the CSP 650 from the CSP to the local receiver 400. In an exemplary implementation, the CSP 650 comprises a “smart” fingerstick apparatus, including at least (i) sufficient onboard processing capability to generate calibration data useful with the local receiver 400 based on signals or data output from the blood sensor 655, and (ii) a data interface to enable transmission of the data to the local receiver 400. In one configuration, the sensor 655 includes a needle or lancet apparatus 657 which draws a sample of the user's blood for the sensor 655 to analyze. Electronic glucose “fingerstick” apparatus (including those with replaceable single-use lancets) and re-usable electronic components are well known in the relevant arts, and accordingly not described further herein. See e.g., U.S. Pat. No. 8,357,107 to Draudt, et al. issued Jan. 22, 2013 and incorporated herein by reference in its entirety, for one example of such technology. The sensor 655 analyzes the extracted blood obtained via the lancet 657 and (via the onboard processing) produces data indicative of a blood glucose level (or at least generates data from which such level may be derived), such data being provided to the communications interface 659 for transfer to the local receiver 400. The transmitted data are then utilized within the local receiver 400 for calibration of the data generated by the implanted sensor 200.

In one variant, the interface 659 comprises a Bluetooth-compliant interface, such that a corresponding Bluetooth interface of the local receiver can “pair” with the CSP 650 to effect transfer of the calibration data wirelessly. Hence, the user with implanted sensor 200 can simply use a fingerstick-based or other type of external calibration data source to periodically (e.g., once weekly) confirm the accuracy and/or update the calibration of the implanted sensor 200 via opportunistic communication between the local receiver 400 (e.g., small profile wristband, fob, etc.) and CSP 650 when convenient for the user. Advantageously, many persons with diabetes possess such electronic fingerstick-based devices, and wireless communication capability is readily added thereto by the manufacturer at little additional cost.

In another variant, the communications interface comprises an IR or optical “LOS” interface such as one compliant with IrDA technology, such that the user need merely establish a line-of-sight path between the emitter of the CSP 650 and the receptor of the local receiver 400, akin to a television remote control. As yet another alternative, a near-field communication (NFC) antenna may be utilized to transfer data wirelessly between the apparatus 400, 650 when placed in close range (i.e., “swiped”). Yet other communication modalities will be recognized by those of ordinary skill given the present disclosure. Additional functionalities of the local receiver 400 and parent platform 600 are described in U.S. patent application Ser. No. 15/368,436 filed Dec. 2, 2016 previously incorporated herein.

It will be appreciated that the architectures shown in FIGS. 3-3B are in no way exclusive of one another, and in fact may be used together (such as at different times and/or via different use cases). For example, CSP 650 can be used in combination with receiver and processor apparatus 450 for opportunistic communication of calibration data. Myriad other permutations of use cases involving one or more of the various components 200, 400, 450, 600, 650, 700 are envisaged by the present disclosure.

FIG. 3C is a functional block diagram illustrating an exemplary implantable sensor apparatus 200 and local receiver and processor apparatus 450 or local receiver apparatus 400 according to one embodiment of the present disclosure. As shown, the sensor apparatus 200 includes a processor 210 (e.g., digital RISC, CISC, and/or DSP device), and/or a microcontroller (not shown), memory 216, software/firmware 218 operative to execute on the processor 210 and stored in e.g., a program memory portion of the processor 210 (not shown), or the memory 216, a mass storage device 220 (e.g., NAND or NOR flash, SSD, etc. to store collected raw or preprocessed data or other data of interest), one or more analyte detectors 226, a wireless interface 228 (e.g., narrowband, PAN such as Bluetooth, or other, described below), and a power supply 230 (e.g., a primary Lithium or rechargeable NiMH or Lithium ion battery). As can be appreciated by those of ordinary skill given the present disclosure, any number of different hardware/software/firmware architectures and component arrangements can be utilized for the sensor apparatus 200 of FIG. 3C, the foregoing being merely illustrative. For instance, a less-capable (processing, and/or data storage-wise) or “thinner” configuration may be used, or additional functionality not shown added (e.g., miniature accelerometer to, inter alia, enable detection of host movement and ambulatory state, orientation, etc.).

### Receiver Apparatus—

Referring now to FIGS. 4-4B, various embodiments of the receiver apparatus 400 and 500 shown in FIGS. 3-3C herein are described in detail.

FIG. 4 depicts a functional block diagram of one embodiment of the receiver and processor apparatus 450, in wireless communication with the analyte sensor 200 of FIG. 3C via the interposed tissue (boundary) 101. As noted previously, the present disclosure contemplates use of partially-implanted (e.g., transcutaneous) or even non-implanted analyte sensor devices, as well as the fully-implanted device (e.g., sensor apparatus 200 of FIG. 2).

As shown in FIG. 4, the receiver/processor apparatus 450 includes a processor 404 (e.g., digital RISC, CISC, and/or DSP device), and/or a microcontroller (not shown), memory 406, software/firmware 408 operative to execute on the processor 404 and stored in e.g., a program memory portion of the processor 404 (not shown), or the memory 406, a mass storage device 420 (e.g., NAND or NOR flash, SSD, etc. to store received raw or preprocessed data, post-processed data, or other data of interest), a wireless interface 416 (e.g., narrowband or other, described below) for communication with the sensor apparatus 200, a communications (e.g., wireless) interface 414 for communication with the network 700 (if desired), a power supply 430 (e.g., NiMH or Lithium ion battery, or other as described below), and a graphical display device 540. The apparatus 450 can optionally include one or more output device(s) 410 for communication of the desired data (e.g., glucose level, rate, trend, battery “low” alerts, etc.) in addition to the display 440. As described in greater detail subsequently herein the output device(s) may include for example visual, audible, and/or tactile (e.g., haptic) modalities or mechanisms, which can be used alone or in concert depending on user context, desired functionality, and receiver and processor apparatus configuration.

FIG. 4A is a functional block diagram showing one embodiment of the local receiver apparatus 400, in wireless communication with the parent platform 600 and the analyte sensor 200 (discussed supra) of FIG. 3C via the interposed tissue (boundary) 101.

Similar to the receiver and processor apparatus 450, the local receiver apparatus 400 includes a processor 404 (e.g., digital RISC, CISC, and/or DSP device), and/or a microcontroller (not shown), memory 406, software/firmware 408 operative to execute on the processor 404 and stored in e.g., a program memory portion of the processor 404 (not shown), or the memory 406, a mass storage device 420 (e.g., NAND or NOR flash, SSD, etc. to store received raw or preprocessed data, post-processed data, or other data of interest), a wireless interface 416 (e.g., narrowband or other, described below) for communication with the sensor apparatus 200, a communications (e.g., wireless) interface 414 for communication with the parent platform 600, and a power supply 430 (e.g., NiMH or Lithium ion battery, or other as described below). The apparatus 400 also includes one or more output device(s) 410 (such as e.g., visual, audible, and/or haptic modalities or alert mechanisms) for communication of the desired data (e.g., glucose level, rate, trend, battery “low” alerts, etc.).

Notably, in the embodiment shown in FIG. 4A, the local receiver 400 lacks a full graphical display device (such as the graphical display device 440 shown in FIG. 4). Exclusion of the foregoing graphical display allows for overall reduced dimensions of the local receiver apparatus 400 making it suitably sized for wear or implantation (such as the exemplary wearable and implantable local receivers discussed supra and described in U.S. patent application Ser. No. 15/368,436 filed Dec. 2, 2016 previously incorporated herein). Rather, the data from the local receiver apparatus 400 is displayed at a full graphical display device 640 associated with the parent platform 600. In some embodiments, the local receiver 400 includes a reduced or limited graphical display (i.e., small graphical display) as one of the output device(s) 410. For example, a wrist wearable local receiver can include a small LCD, or even a capacitive touch screen for sending alerts and/or other information to the user as well as receiving input from the user.

FIG. 4B is a functional block diagram illustrating yet a further exemplary embodiment of the local receiver apparatus 400 of FIG. 4A, wherein the local receiver apparatus is implanted within a host and communicates wirelessly with both a blood analyte (e.g., glucose) sensor and a parent platform. For instance, the apparatus 400 can be implanted subcutaneously, and the user output device 420 can comprise a haptic apparatus that encodes signals perceptible by the user (i.e., under their skin). The wireless interface 416 can comprise e.g., the aforementioned 433 MHz narrowband system which is effective at propagating through human tissue, and hence the sensor 200 can communicate directly with the local receiver in vivo., or alternatively other types of interfaces with sufficient RF energy propagation through tissue such as a Bluetooth ISM-band (approximately 2.45 GHz) transceiver, or ZigBee PAN transceiver at approximately 915 MHz or 2.4 GHz.

Moreover, the implanted local receiver 400 can be configured such that the power supply 430 is passively activated (e.g., through incident RF energy, including that of the sensor 200,), thereby obviating explants of the device (except for component failure).

The receiver apparatus 400 of FIG. 4E can also include a second wireless interface 414 for communication to the parent platform 600, such as a Bluetooth IC or similar.

Additional configurations and functionality of the various receiver apparatus are described in U.S. patent application Ser. No. 15/368,436 filed Dec. 2, 2016 previously incorporated herein.

### Operational Methods—

Referring now to FIGS. 5-5A, exemplary embodiments of the methods of operating the local receiver apparatus 400 and the receiver and processor apparatus 450 (and analyte sensing systems generally) are described in detail.

FIG. 5 is a logical flow diagram illustrating one exemplary embodiment of a method 500 of operating a local receiving device and/or a dedicated receiver and processor apparatus for blood analyte measurement according to the present disclosure.

As shown, the method 500 begins with the user or clinician enabling the sensor (e.g., implanted device 200 of FIG. 2, or other) per step 502. In the case of the implantable sensor of FIG. 2, the sensor is enabled, implanted in the host (such as via the procedures described in U.S. patent application Ser. No. 14/982,346 filed Dec. 29, 2015 previously incorporated herein), and tested as part of step 502.

Next, the receiver apparatus 400, 450 (e.g., any of those of FIGS. 4-4B herein) is enabled, and maintained within communications range of the sensor apparatus, per step 504. As noted supra, the exemplary embodiment of the sensor apparatus uses a 433 MHz narrowband RF transmitter (such frequency having good signal transmission characteristics through human tissue), and hence has a communications range, dependent on transmission power, of at least several feet. Hence, in one implementation of the method step 504, the host/user merely needs to keep the receiver 400, 450 within arm's reach, or somewhere on their body personally.

Per step 506, the enabled sensor 200 communicates data wirelessly to the receiver apparatus 400, 450, such as on a periodic, event-driven, continuous, or other basis. Note that the transmission and reception frequencies or schedule need not necessarily coincide completely. For instance, the transmitter of the sensor apparatus 200 may transmit according to a prescribed periodicity or frequency, while the receiver 400, 450 may utilize a less frequent sampling of the transmissions.

Per step 508 of the method 500, the received sensor data are processed to calculate blood analyte level, and any related parameters or data derived therefrom. Such processing may occur when the data are received, or collectively in one or more aggregations or batches of data (e.g., sensor data collected or received over a prescribed time period).

Per step 510, the calculated blood analyte level (e.g., glucose concentration in e.g., mg/dL or mmol/L) is output to the user in a cognizable form, such as visually, via haptic apparatus, audibly, and/or yet other means, as described elsewhere herein. Similarly, other information (such as, e.g., trend of the blood glucose level, ROC, and/or alert notifications discussed in detail infra) may be output from the receiver 400, 450 via the same or different cognizable medium. For instance, in one embodiment, the blood glucose level is displayed on a display device of the local receiver as a sequence of numbers (e.g., “123”), while alerts or warnings are output as audible tones or “chirps,” and/or haptic pulses to the user via the local receiver's contact with their skin. Numerous permutations of the foregoing will be appreciated by those of ordinary skill given the present disclosure.

Specifically with use of the local receiver 400, the method 500 further includes determining whether the parent platform 600 (e.g., the user's more fully-functioned tablet, smartphone, etc.) is “communicative” with the local receiver 400 (per step 518). This determination may be made actively or passively, periodically or based on an event, and directly or indirectly.

Returning to FIG. 5, when communications between the local receiver 400 and the parent platform 600 are enabled per step 512, the local receiver and parent platform handshake (e.g., pair according to a Bluetooth pairing protocol, with the local receiver as the slave, and the parent as the master). In one implementation, the master/slave architecture inherent in the Bluetooth topology is advantageously leveraged to enable multiple simultaneous pairings between a single parent platform and two or more local receivers (e.g., associated with two or more respective individuals), such that “group updates” can be performed substantially simultaneously.

At step 520, processed and/or raw data from the local receiver 400 is transmitted to the parent platform 600. Per step 522 of the method 500, the local receiver(s) receive the configuration and/or calibration data as applicable from the parent platform, and per step 524, utilize the received data to confirm calibration of the sensor e.g., through comparison of “fingerstick” or blood glucose monitor (BGM) values entered by the user via the parent platform software UI.

Per step 526, the configuration of the local receiver (e.g., the alert setting values, alert logic or hierarchy such as “haptic then visual then audible”, etc. discussed in detail infra) is also updated as needed.

Alternatively, if the parent platform is not “communicative” (e.g., outside range, busy, preempted, etc.) per step 512 of the method, operation of the local receiver 400 is continued (i.e., periodic or continuous receipt and processing of sensor data).

FIG. 5A is a logical flow diagram illustrating one exemplary implementation of the sensor data processing and output methodology 511 for the local receiver 400/parent platform 600 or the receiver and processor apparatus 450 according to the method 500 of

FIG. 5. As shown, the method 511 in one embodiment includes first receiving the wireless data transmissions from the (implanted) sensor 200 per step 515. Next, per step 517, the received wireless signals are processed (see the exemplary method of FIG. 5B, discussed below), and the processed data stored (step 521). Exemplary implementations of sensor data receipt and demodulation/unscrambling methodology for the receivers 400, 450 are described in GLYS. 010 previously incorporated herein.

Next, blood analyte level is calculated per step 523, and also other parameters of interest if any (such as real-time trend and/or rate of change) are calculated per step 525. The calculated values from steps 523, 525 are then converted per step 527 to a prescribed output format (e.g., a graphic rendering of a numeric value, a graphic display of a trend arrow, a sequence of haptic vibrations, etc.) consistent with the selected/configured output modality. The converted values or indications are then output to the user in the appropriate modality/modalities per step 529, such as via the user interface (UI) discussed in detail infra.

### User Interface (UI) Apparatus—

As discussed supra, each of the local receiver apparatus 400, the parent platform device 600, and the dedicated receiver and processor apparatus 450 may be configured with a user interface (UI). FIG. 6A illustrates an exemplary configuration for the dedicated receiver and processor apparatus 450 (including the full graphical display 440 for display of the UI), while FIG. 6B depicts an exemplary configuration for the local receiver apparatus (including an output device 410 such as e.g., a reduced graphical display for display of the UI), and the parent platform 600 (including the full graphical display 640 for display of the UI).

In each of the above exemplary configurations, the apparatus can further include audible and/or haptic alert mechanisms, as well as other visual alert mechanism (such as e.g., indicator lights separate from the graphical display). Further, each of the apparatus includes a mechanism (such as e.g., buttons, a keyboard, a capacitive touch screen, etc.) for receipt of user input such as e.g., user parameters or preferences or a user response to an alert.

It will be appreciated that for either of the exemplary configurations shown in FIGS. 6A and 6B (as well as other exemplary configurations discussed herein), substantially similar methodology can be employed for displaying data and receiving user input via the UI.

### UI Configurations and Methods—

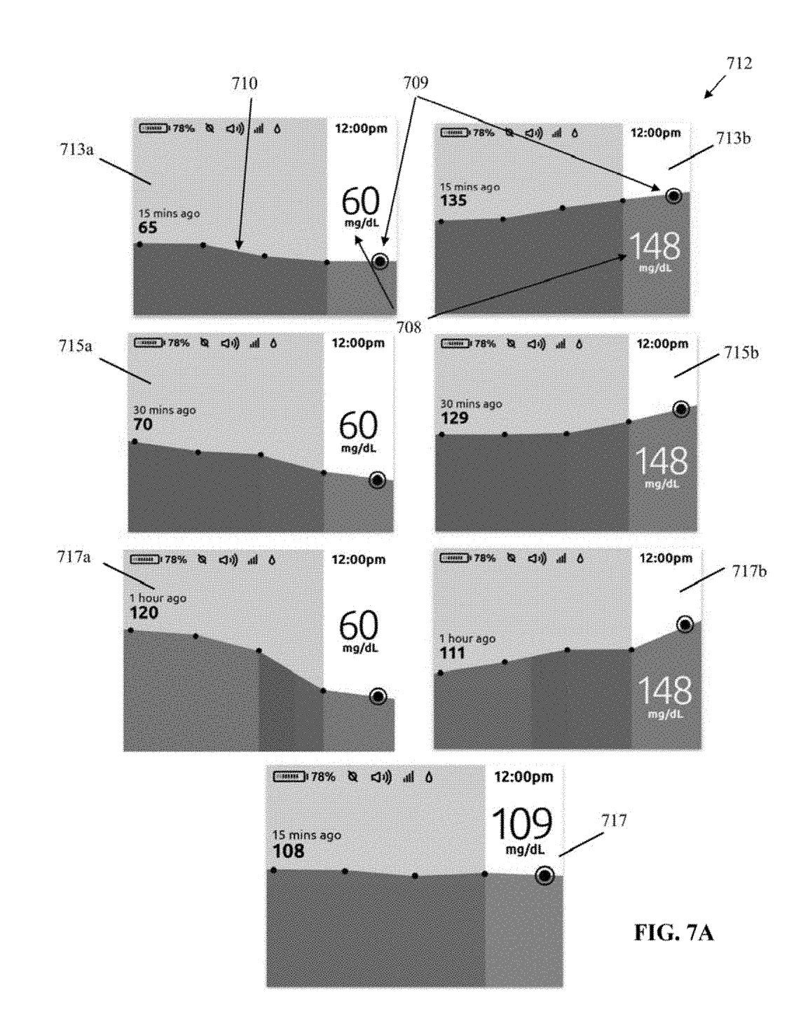

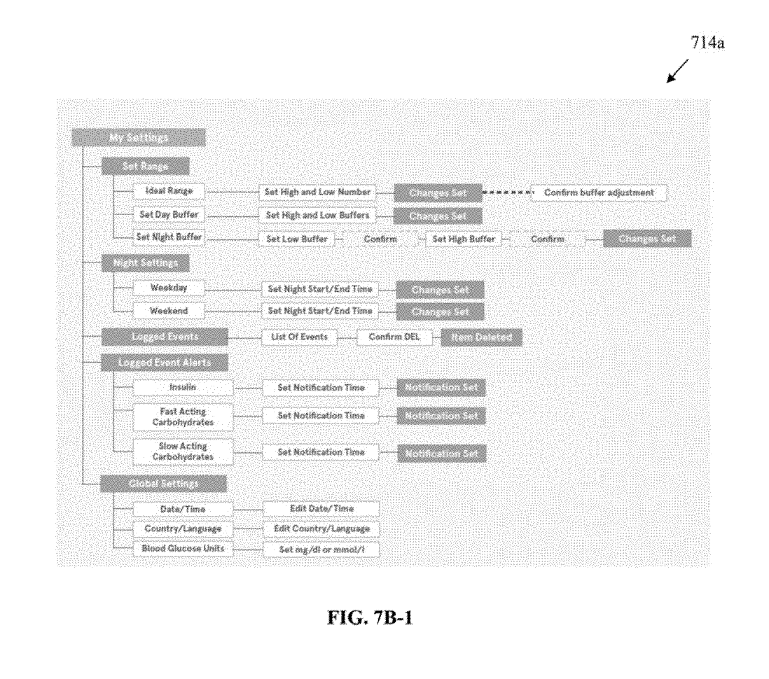

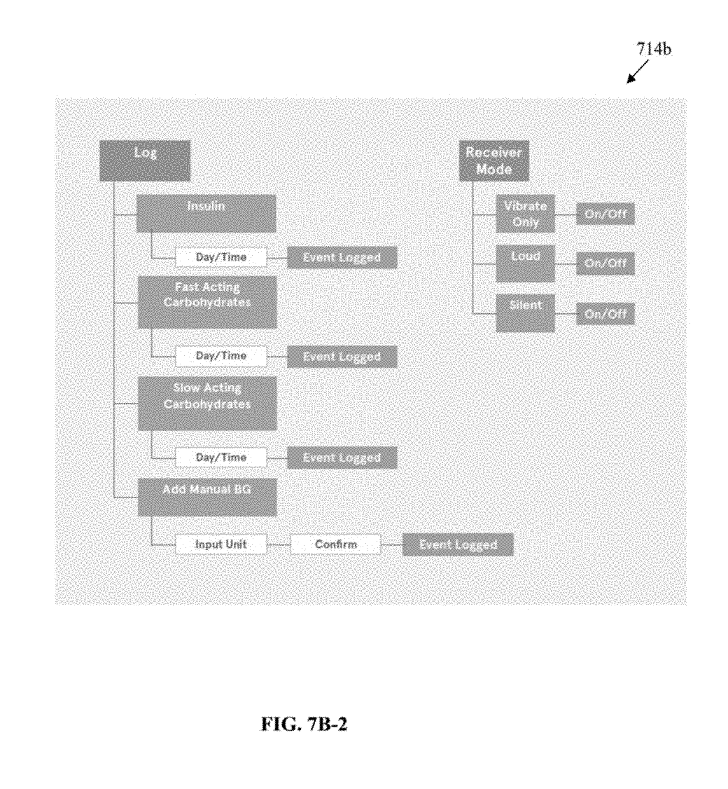

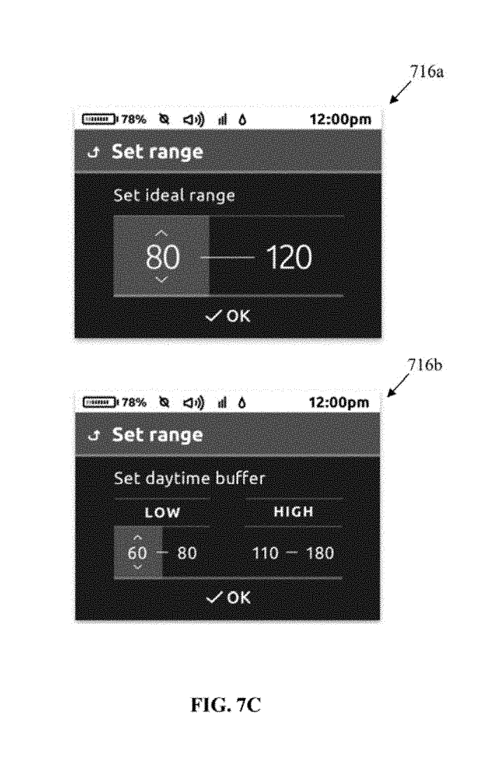

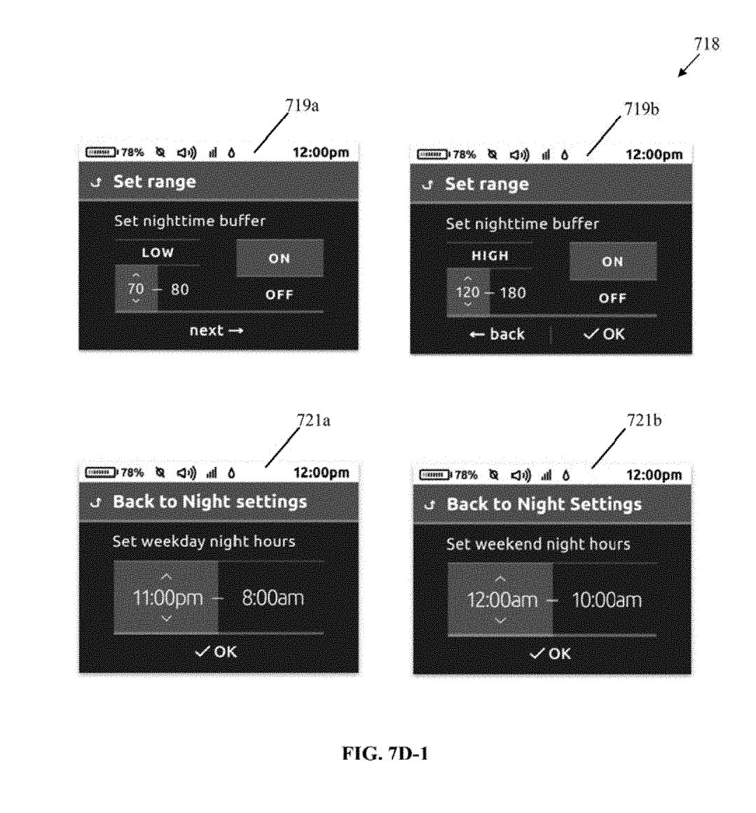

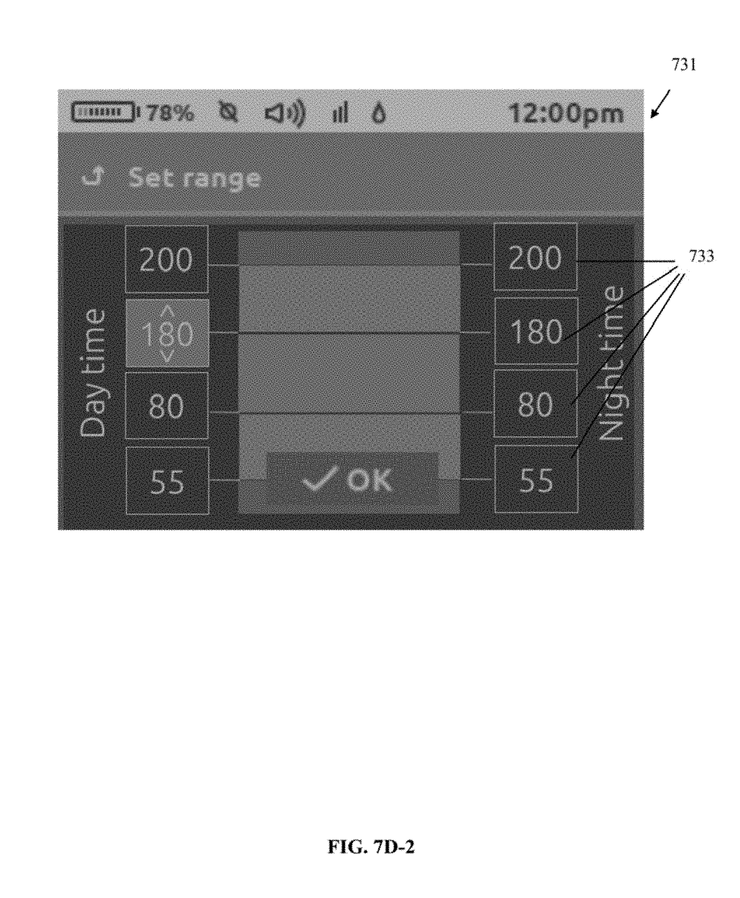

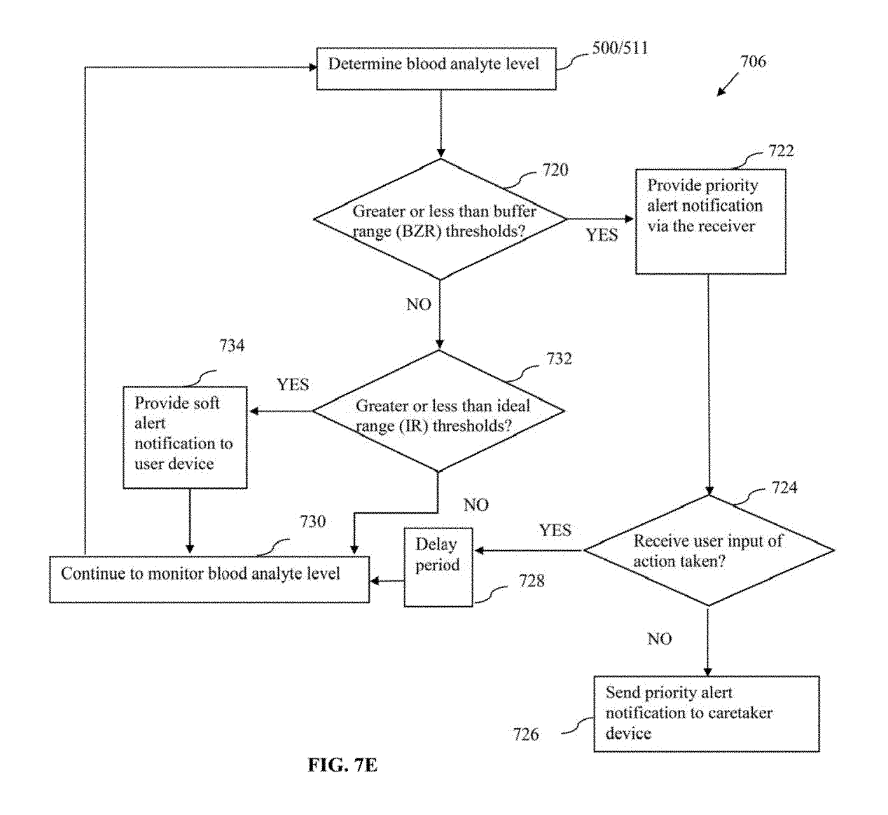

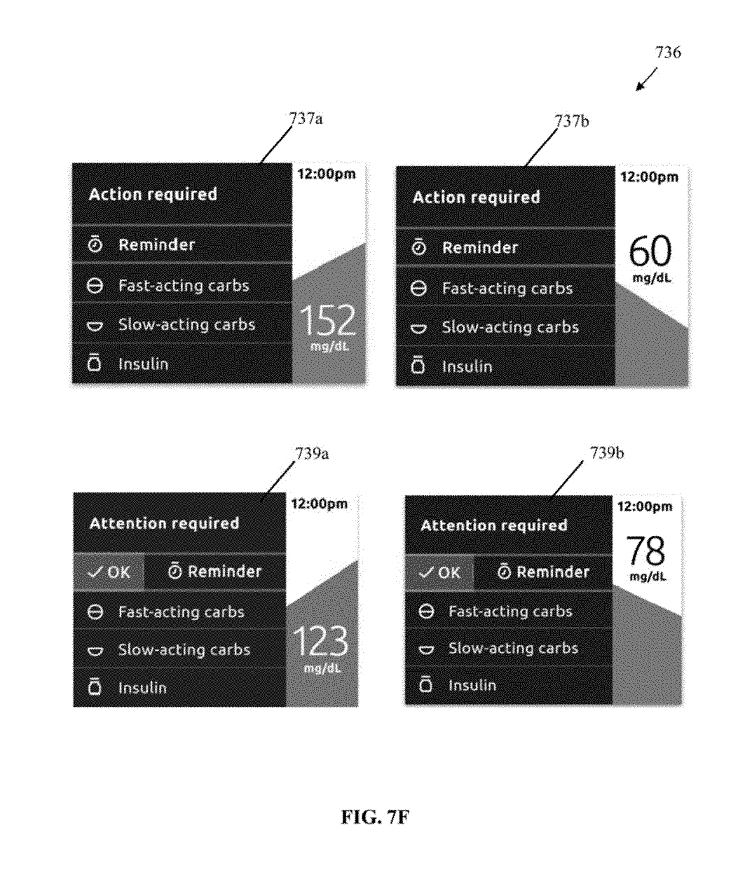

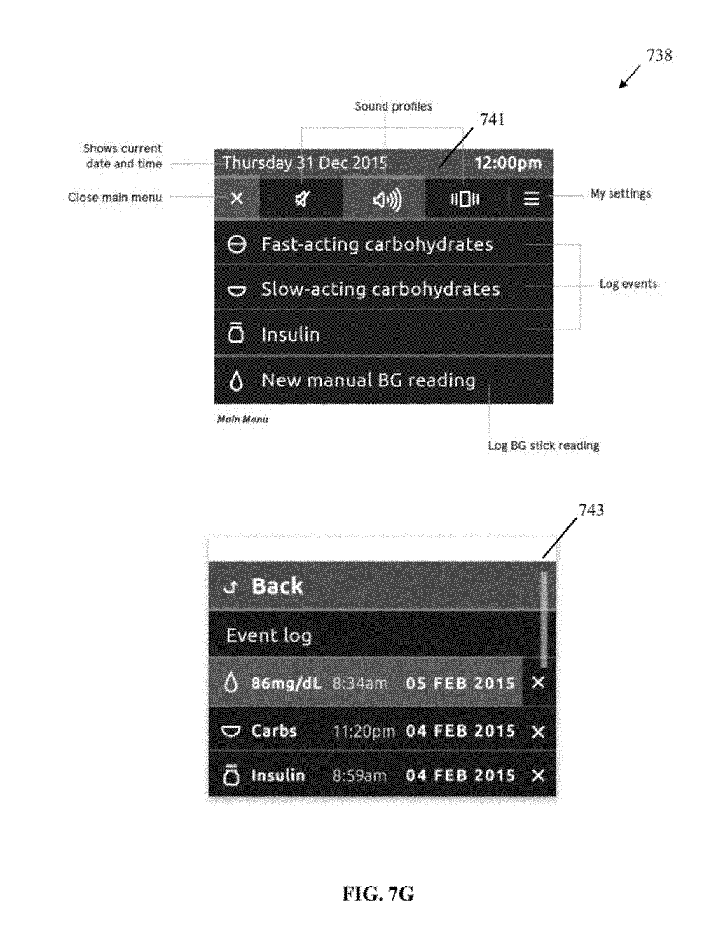

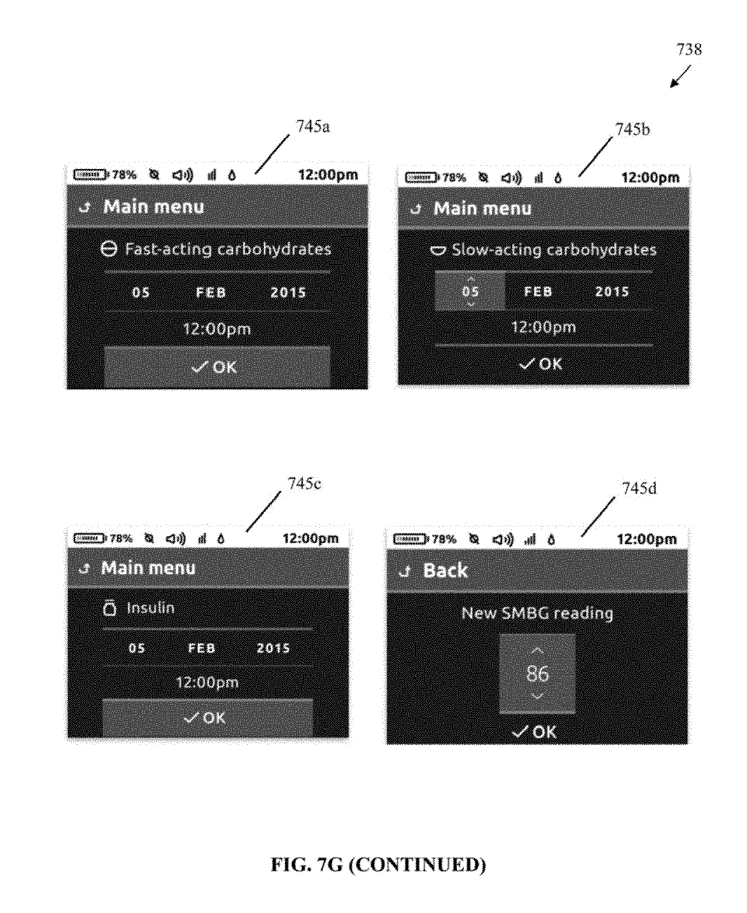

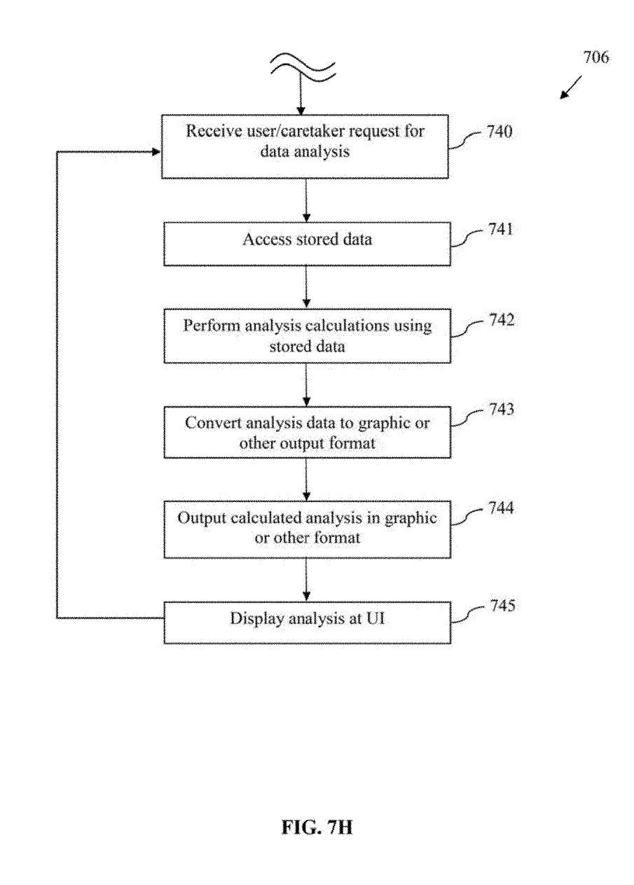

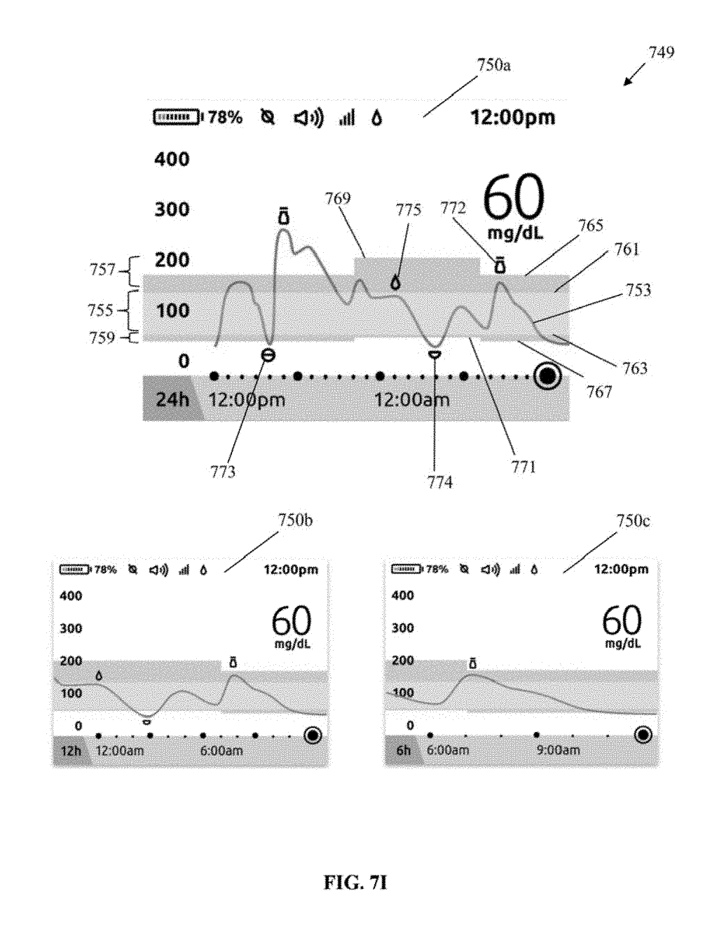

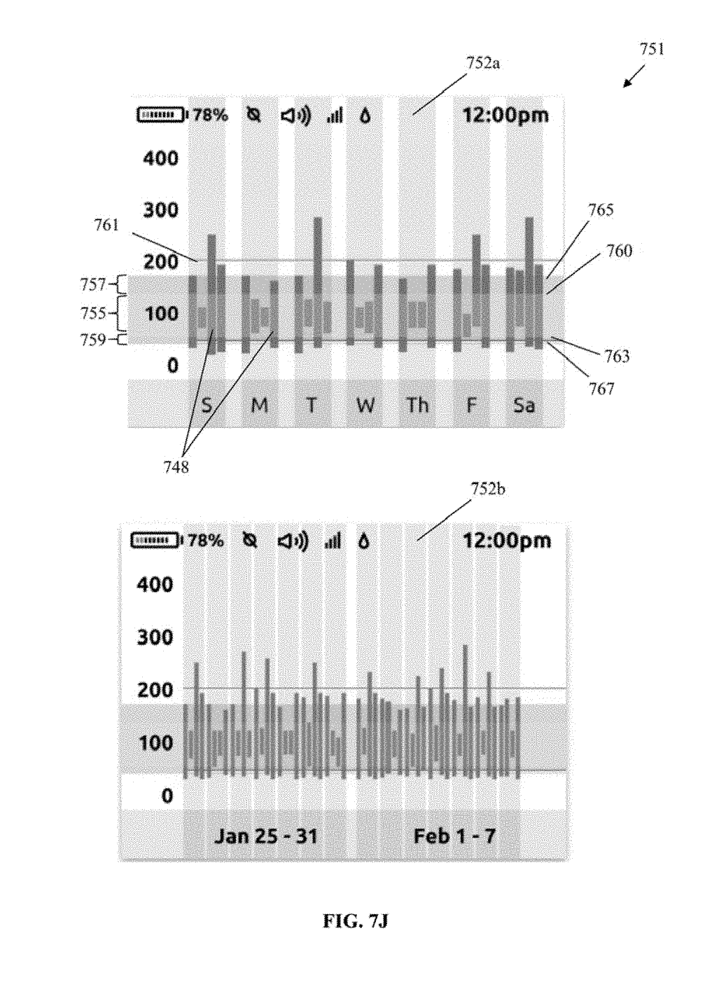

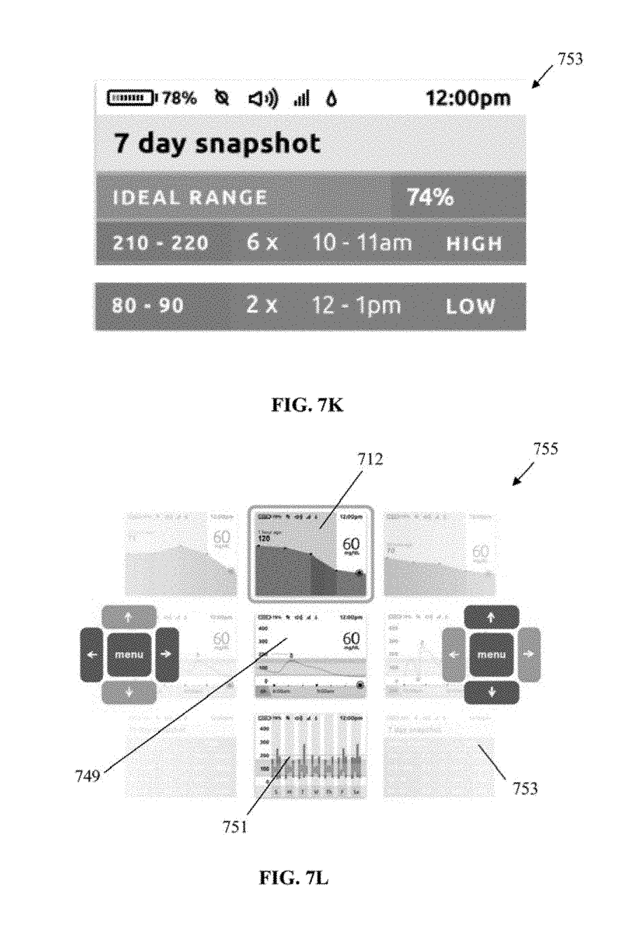

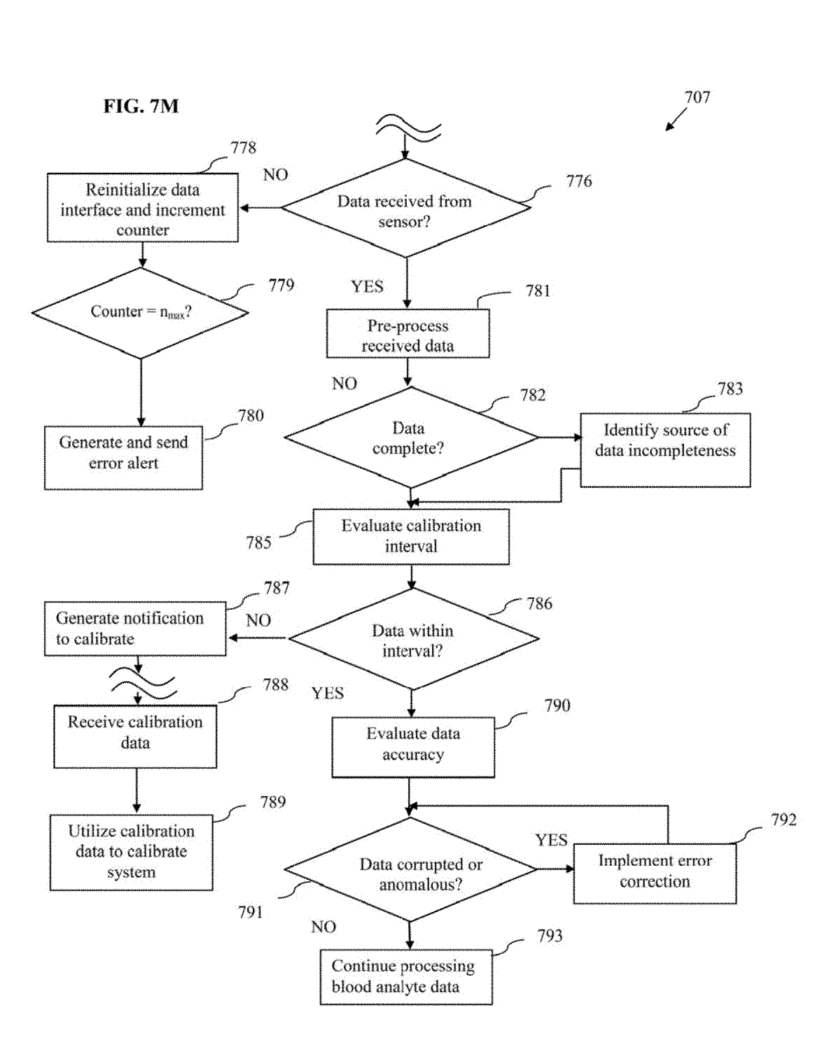

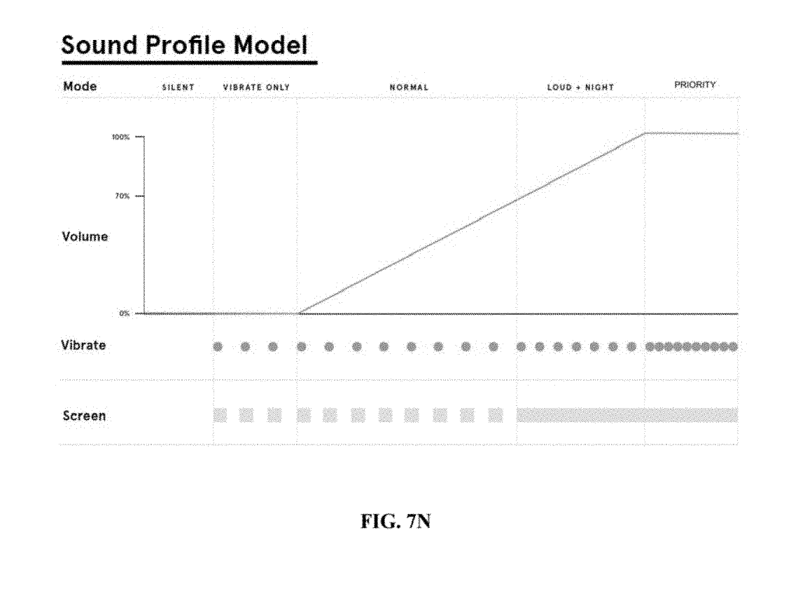

Referring now to FIGS. 7-7N, exemplary embodiments of the configurations and methods of interfacing with a user are described in detail. As will be described in greater detail, such user interfaces also may provide functionality for processing or calculating data, identifying and diagnosing data errors (such as e.g., a need for calibration of the sensor, out-of-range data, identification of non-functional sensor elements, etc.), and other functions.

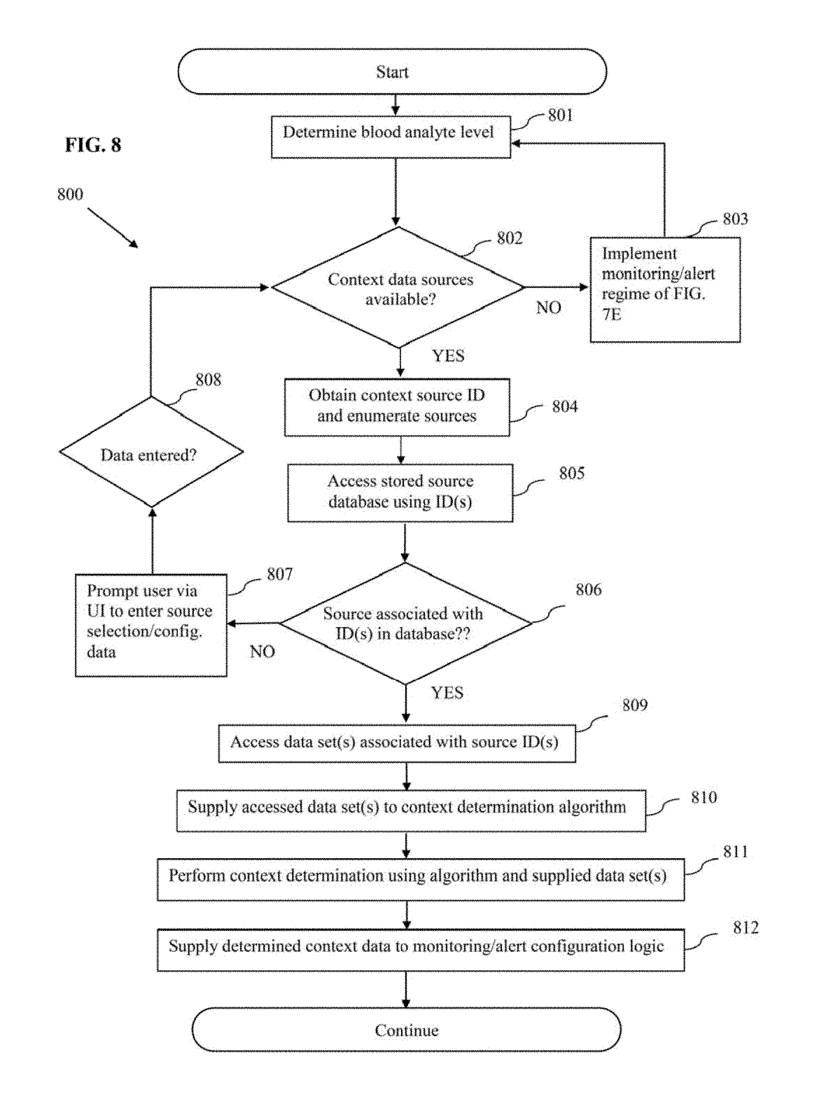

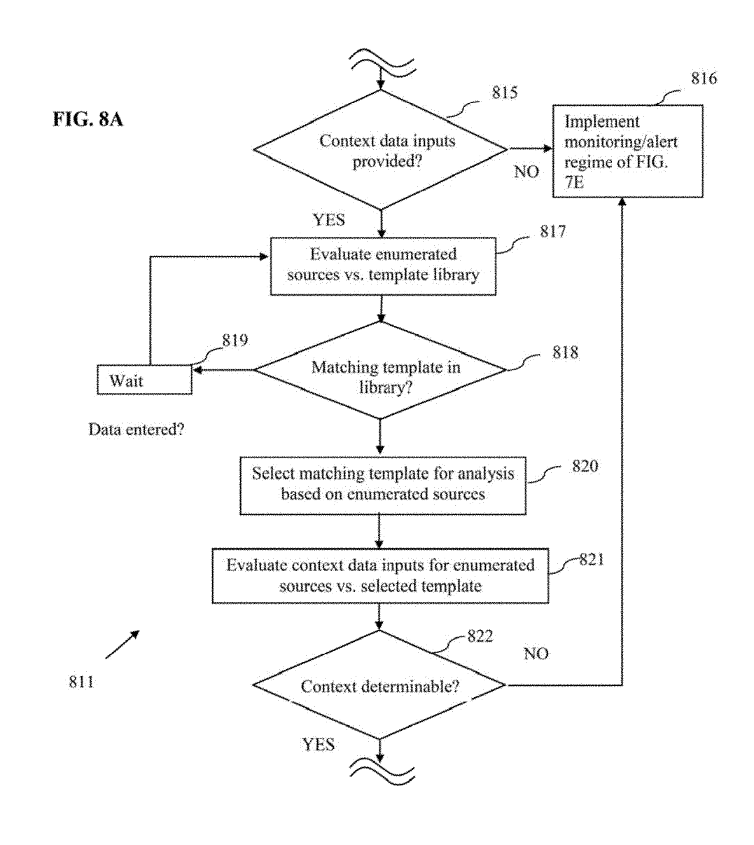

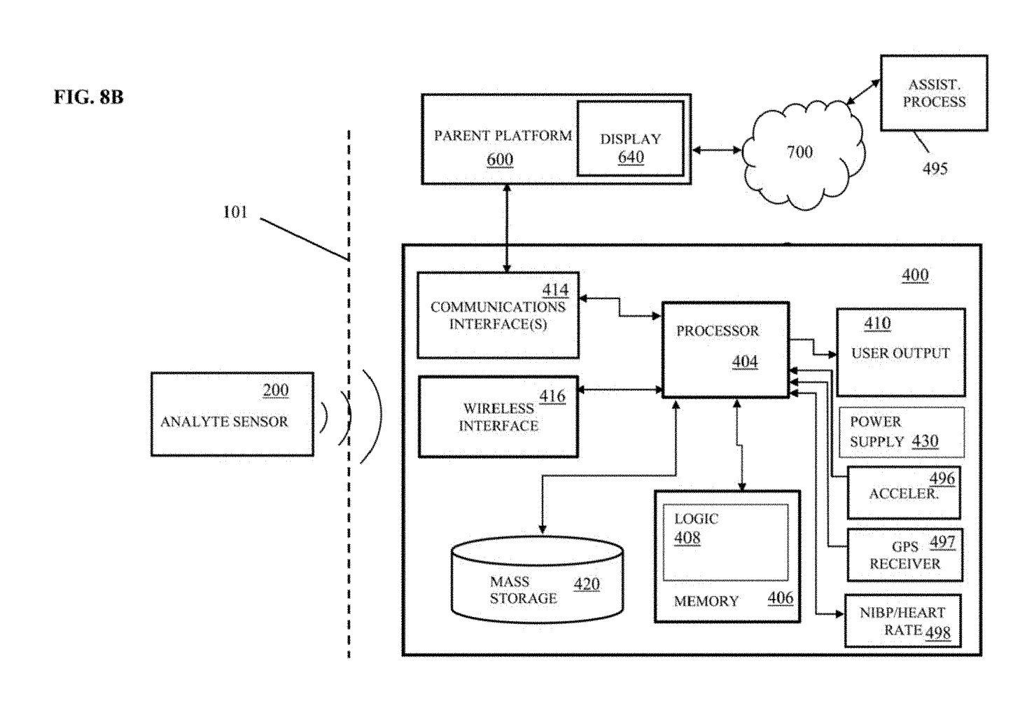

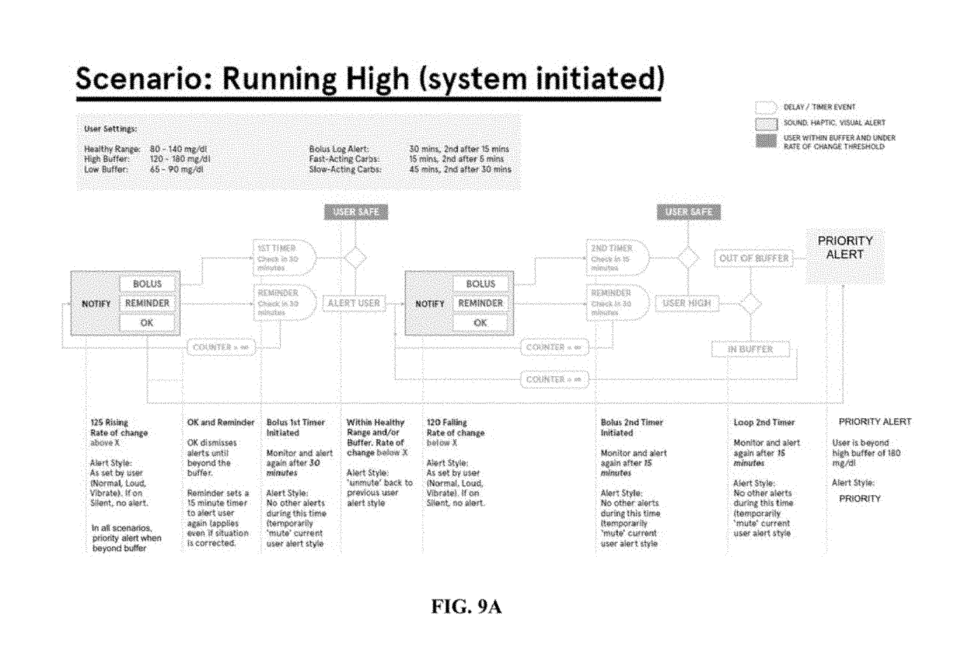

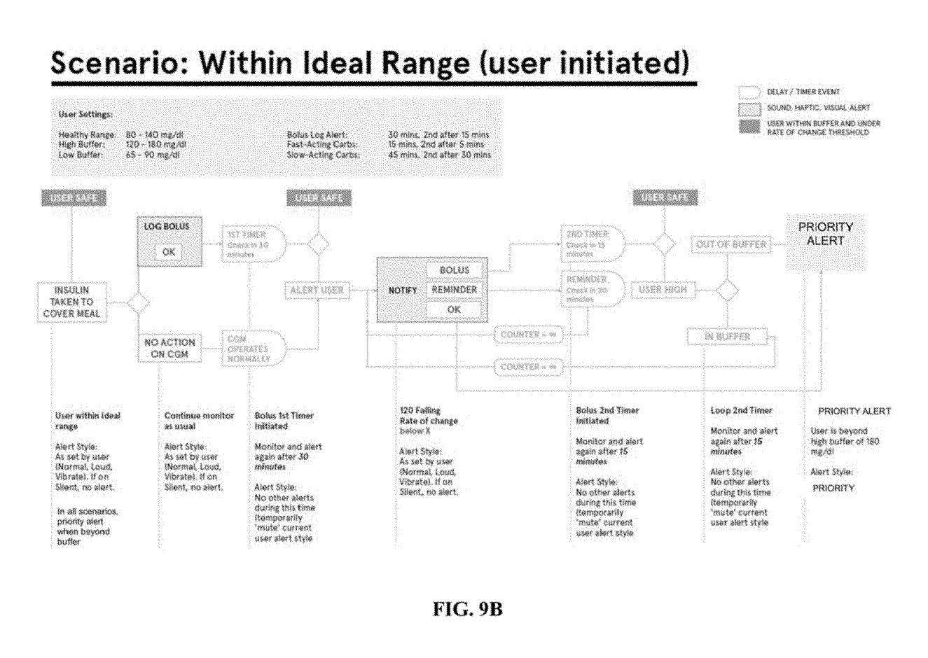

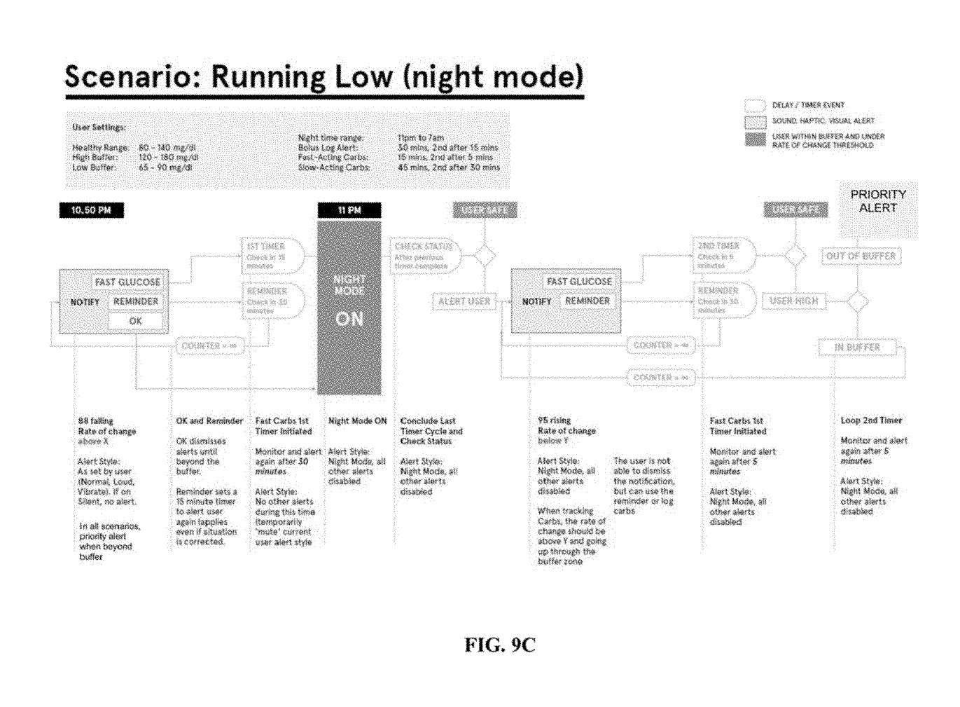

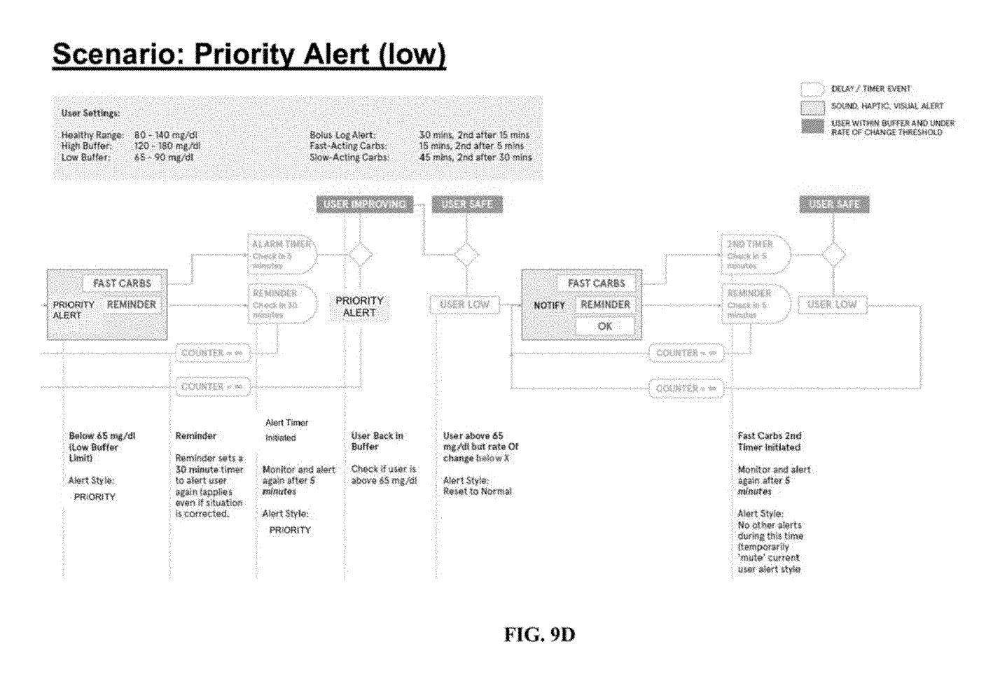

Further, exemplary use-cases or scenarios for the blood analyte detection system, including the aforementioned UI functions, are shown and described with respect to FIGS. 8A-8D discussed infra.

FIG. 7 is a logical flow diagram illustrating one exemplary embodiment of a method 700 of operating a UI of a local receiver apparatus 400, receiver and processor apparatus 450 and/or a parent platform 600 for user-customized blood analyte measurement. As shown, the method 700 begins with receiving and storing user-specified parameters via the UI, essentially comprising “set up” of the blood analyte system per step 702.

After receiving and storing the user input, the local receiver 400 or receiver/processor 450 receives, processes, and stores sensor data in order to calculate and output a current blood analyte level, real-time trend data, and/or ROC data (via e.g., the exemplary methods 500 and 511 shown in FIGS. 5 and 5A, respectively). The data are then displayed on the UI (step 704), such as via the formats or configurations shown in FIG. 7A, described in detail below.

After or concurrently with display of the current blood analyte data per step 704, the current blood analyte level is assessed, and alerts are provided to the user as necessary via the receiver/apparatus 400, 450 (e.g., via the UI associated with the receiver) per step 705.

In addition to provision of alerts (via step 705), the method 700 further includes selectively displaying analyses of blood analyte level at step 706, such as for showing trends (such as via the exemplary method depicted in FIG. 7H discussed infra).

In addition to provision of alerts (via step/method 706) and selective display of trend or other analyses of blood analyte level per step 706, the method 700 of FIG. 7 further includes identification of data errors per step 707 (such as via the exemplary method depicted in FIG. 7M, discussed infra). Such identification of data errors can also be utilized to enable mitigation of such data errors during operation of the blood analyte detection system; e.g., to prevent them from skewing or disproportionately affecting data or indications output by the system to the user or other entity.

In some embodiments, the UI is further configured to receive user-specified high and low thresholds for a non-imminently dangerous blood analyte level; i.e., a level analyte that is of concern but is not yet dangerous. In other words, the UI allows the user to specify high and low buffer zone ranges (i.e., “Buffer Zone Range” (BZR)) that are proximate to the IR high and low thresholds, whether symmetrically or non-symmetrically. Thus, a blood analyte level within the BZR is one which is approaching (but is not yet) a potential health risk to the user. In such embodiments, the user-specified BZR can advantageously enable provision of early, non-emergent alert notifications (i.e., “soft alerts”) to the user via the receiver 400, 450 (and/or the parent platform 600), thereby warning the user of potential health risks prior to sending an acute or priority alert. In some cases, a user can mitigate a potential health risk, and/or obviate more extreme intervention, by promptly taking an action to respond to the soft alert. Specific methods for provision of alerts are discussed in detail infra.

Any region lying outside of the above-described IR and BZRs is in one implementation a “Priority Range” (PR), which characterizes a blood analyte level that may pose an immediate or otherwise important danger to the user's health if no action is taken.

In the exemplary variants described herein, the high and low BZRs are user-defined ranges that lie outside of the aforementioned IR. Specifically, the high BZR is a blood analyte level range above the IR high threshold, and the low BZR is a blood analyte level range below the IR low threshold. In other words, the high threshold of the IR is equal to the low threshold of the high BZR, and the low threshold of the IR is equal to the high threshold of the low BZR.

For ease of implementation, a preferred arrangement of the high BZR is such that it is contiguous with the IR high threshold and a preferred arrangement of the low BZR is such that it is contiguous with the IR low threshold. However, it will be appreciated that the BZRs need not be arranged so contiguously, that is, there may be a gap between each of the high and low thresholds and the high and low BZRs, respectively, and benefits of the invention may still be provided.

It will be appreciated that while the IR, BZRs, and PRs are each described with respect to the exemplary embodiments as being “binary” and linear in nature (i.e., the blood glucose level either falls within the range, in which case an action is taken by the system logic without regard to proximity to a prescribed value, or does not fall within the range, in which no action is taken), one or more of these ranges can be configured to be non-binary or non-linear in nature. For instance, a blood glucose level falling just above the IR within the high BZR and displaying a comparatively low rate of change may warrant a less invasive/emergent monitoring or alert regime than a value closer to the upper bound of the high BZR.

Moreover, such non-linear monitoring and alert regimes may be asymmetrical with respect to the upper and lower bounds of any given range (or set of ranges). For instance, it may be known that certain physiologic processes within the host being (e.g., human) occur differently, or at different rates, depending on the absolute level of the analyte. Stated simply, a “low low” may be more significant than a “high high,” such as in the case of hypoglycemic states which can readily result in host mortality if not addressed. Accordingly, a rate of change (ROC) associated with a decreasing blood glucose level can be monitored and alerted more stringently than a similar rate of change for an increasing blood glucose level, whether in the low BZR or the high BZR. Likewise, a given proximity (e.g., in percent, or absolute mg/dl) to the lower bound of the low BZR can be monitored and alerted more stringently than a similar proximity to the higher bound of the high BZR.

In an alternate variant, the high and low BZRs for blood analyte level are configured to fall wholly within the aforementioned IR, and are defined (respectively) by a range between the IR high threshold and a high BZR threshold (a blood analyte level less than the high IR threshold) and a range between the IR low threshold and a low BZR threshold (a blood analyte level greater than the IR low threshold). Thus, in the former variant, upper and lower limits of the BZR are variable, while in the latter variant upper and lower limits of the BZR are static (i.e., defined by upper and lower limits of the IR). It will be appreciated that the foregoing variants may be alternately or selectively implemented dependent upon the specific medical condition (and type of analyte) monitored and/or managed by the analyte detection system.

The BZRs may also be configured to fall only partly within the IR, such as in a discontinuous or differentiated fashion depending on whether the blood analyte level is within or outside the IR. For instance, if the IR is 80-120 mg/dl, the upper BZR may run from 110 to 130 mg/dl, with the first 10 mg/dl falling within the IR, and the second 10 mg/dl falling above the IR. The monitoring and/or alert regimes implemented by the computerized logic of the system disclosed herein may likewise be differentiated; e.g., “softer” monitoring and/or alert rules may be applied within the 10 mg/dl band within the IR.

In some embodiments, in addition to receiving user-specified parameters for the foregoing high and low buffer zones, the UI further enables alternate threshold settings of the BZR dependent on time of day, day of the week, or other temporal context. In one example, it may be desirable to set generally wider BZRs during a period of user sleep (e.g., “Night Buffer”), and generally narrower BZRs during a period when the user is normally awake (e.g., “Day Buffer”). Thus, out-of-range alert notifications provided by the receiver 400, 450 can be substantially decreased during a time when the user is normally sleeping, as the receiver 400, 450 instead provides soft alerts. In other words, a sensitivity of the receiver can be decreased at night and increased during the day so as to provide pre-warning and warning notifications (as necessary) without overly disrupting the user during a period in which they do not want to be disturbed (e.g., while sleeping), and also at times during which few transients in blood analyte level are anticipated due to lack of ingestion of food or caloric drink, lower user metabolic and heart rate, etc.

Alternatively, it may be desirable to set generally narrower BZRs during a period of sleep for certain users (e.g., “Night Buffer”), and generally wider BZRs during a period when the user is normally awake (e.g., “Day Buffer”). Thus, priority alert notifications provided by the receiver 400, 450 can be substantially increased or provided sooner during a time when the user is sleeping. In other words, a sensitivity of the receiver can be increased at night and decreased during the day so as to provide priority alerts to the user more quickly when asleep, thereby allowing the user sufficient time to “wake-up” and respond to the potentially dangerous blood analyte level condition. For instance, if the user is a very deep sleeper, and requires significant audible, visual, and/or haptic stimulation to wake up and become cognitive (and take corrective action), a more stringent night monitoring and alert regime may be beneficial to avoid blood glucose level excursions from reaching a crisis point. Moreover, hypoglycemia can in some individuals manifest itself in user confusion, lightheadedness, blurred vision, etc., which can further impair the user's ability to take corrective action, thereby making the promptness of the alert function that much more necessary; i.e., if the alert is too latent, the user's ability to intervene may be compromised.

It will also be appreciated that the same user may make use of both of the foregoing day/night sensitivity regimes (i.e., high-day/low-night, and low-day/high-night), depending on factors such as the user's medical or treatment context. For instance, during periods of normal health and cognitive function, the first regime may be used, such use based on the fact that the user wakes easily, and is anticipated to have few BG level excursions at night. However, when that same user has say the flu, or other condition which might affect their cognitive “recovery time” (such as ingestion of medications before bed to help the user sleep with the symptoms that accompany the flu), the second sensitivity regime (i.e., low-day/high-night), or even a hybrid regime (e.g., high-day/high-night) may be selected by the user for use during that night, or a series of successive nights.

More generally, it will be appreciated that the foregoing examples may be implemented alternately or in combination depending upon the specific medical condition, type of analyte monitored by the analyte detection system, historical data or behavior associated with the user or a class of individuals to which the user belongs, and/or user-specific preferences. For example, for many users having a diabetic condition, the user's BG level normally drops at night (as the user sleeping and does not ingest food). Therefore, in management of such diabetic condition, it can be desirable to set a narrower high BZR and a wider low BZR during the day when BG level is more likely to increase, and a wider high BZR and a narrower low BZR at night when BG level is more likely to decrease. In this example, the nighttime sensitivity of the receiver to low BG levels (i.e., less than the IR low threshold) is increased and sensitivity of the receiver to high BG levels (i.e., higher than the IR high threshold) is decreased. Further, in the foregoing example, a priority alert notification for a potentially dangerous low BG level is provided by the receiver/UI to the user more quickly at night than during the day, thereby allowing the user more time to wake up and take action (such as e.g., intake of carbohydrates) to manage the low BG level condition. Furthermore, prority alerts associated with a high BG level are reduced (e.g., instead providing soft alerts, with wider tolerance), thereby limiting disruption to the user's sleep.

Referring now to FIG. 7A, the UI displays 712 depicted therein include a series of exemplary graphical elements for display of current blood analyte levels. As further explained below, the exemplary display elements are chosen for ease of reading by the user (including, inter alia, a high degree of color differentiation and contrast), as well as intuitiveness and easy cognitive recognition (i.e., a quick glance by the user without necessarily cognitively recognizing the numbers or text can still provide useful information in terms of trend and the like). Specifically, as described in greater detail below, the exemplary color scheme and positioning of data elements are chosen so as to (i) provide the user with instant recognition of where the blood glucose level falls with respect to certain prescribed “buffer zones” or other ranges or values of interest; and (ii) collectively provide such cognitive recognition in a temporal aspect; i.e., to differentiate time periods, with a first color and/or intensity being chosen to indicate the most recent portion of the graph, and a similar but distinguishable color/intensity used to indicate a less recent portion,. That way, the user's eye is immediately focused on the most relevant portion of the graph (i.e., the current temporal period), with an easily readable blood glucose level displayed within that region.

As depicted in FIG. 7A, each of the exemplary blood analyte level display outputs 712 includes a current blood analyte level (e.g., a numeric indicator 708 and a visual marker 709), and real-time trend data (e.g., a trend line 710), ROC data (e.g. a slope of the trend line). Moreover, an indication of a user-defined range (e.g., Ideal Range (IR), Buffer Zone Range (BZR), and Priority Range (PR) discussed infra) within which the the blood analyte level is located is provided based on coloration of the area under the trend line.

For example, displays 713a, 715a, and 717a of FIG. 7A illustrate exemplary outputs where a user's blood analyte level is 60 mg/dL and trending downward (i.e., decreasing), while displays 713b, 715b, and 717b illustrate display outputs where the user's blood analyte level is 148 mg/dL and trending upward (i.e., increasing).

In another example, display 717 illustrates an exemplary output where the user's blood analyte level is 109 mg/dL and stable.

The current trend data can be selectively viewed at various periods of recent history such as e.g., 15 min. (in display output 713a, 713b, 717), 30 min. (in display output 715a, 715b), or 60 min. (in display output 717a, 717b). Further, the user-specified blood glucose level ranges can be indicated by a coloration, intensity, and/or apparent “texture” or fill of the area under the trend line to provide the user with an immediately identifiable blood analyte level visual reference. In one specific example, values falling within the IR is indicated by a blue coloration (as in displays 717, 717a and 717b of FIG. 7A), the BZR is indicated by a purple coloration (as in displays 715a, 715b, and 717b), and the PR is indicated by an orange or red coloration (as in displays 713a, 713b, 715a, 715b, 717a, and 717b). Obviously, other readily identified “progressive” color or intensity schemes may be used, such a green-yellow-red, white-grey-black, etc.

Also as shown in the displays of FIG. 7A, the area above and below the trend line 710 is also varied in intensity in the current or most recent temporal period, to provide further differentiation (i.e., use of white versus grey above the trend line, and brighter coloration—depending on glucose level—below the trend line).

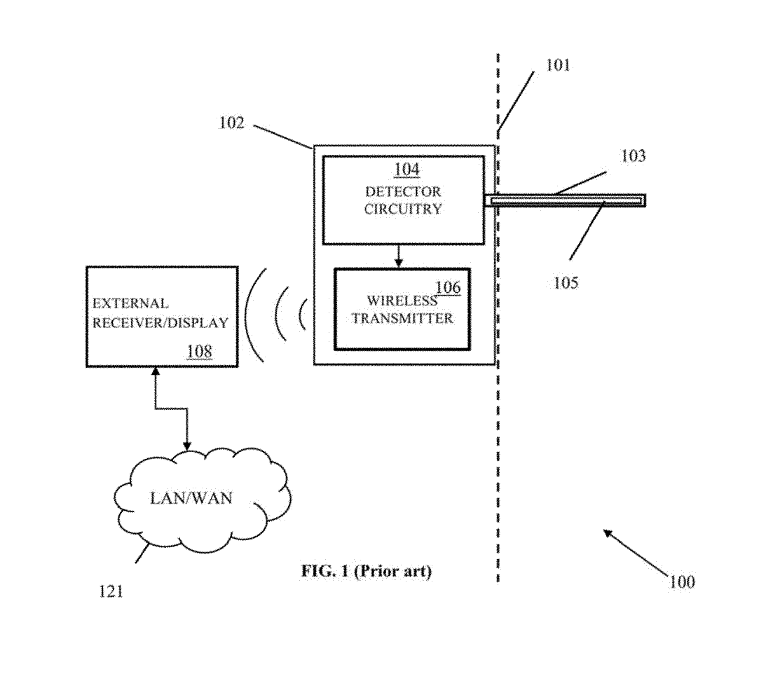

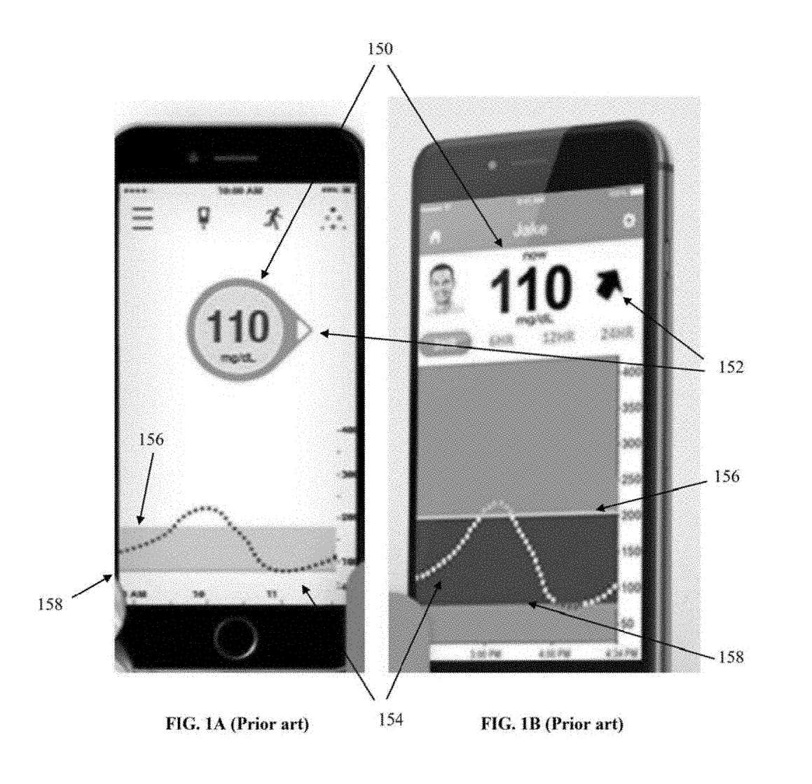

The logical “tree” diagrams of FIGS. 7B-1 and 7B-2 (i.e., “My Settings” tree diagram 714a and a “Log/Receiver Mode” tree diagram 714b, respectively) include exemplary functions relating to user-specified parameters which may be accessed via the UI. As shown in FIGS. 7B-1 and 7B-2, the exemplary logical structure of the software/firmware supporting the UI is configured to: (i) receive user-specified parameters for blood analyte level ranges (e.g., IR, BZR, and/or PR) specific to a time of day and/or a weekday vs. weekend day, (ii) enable a user to log user action/response events, (iii) enable a user to schedule health management-related notifications, as well as (iv) enable various global device settings (e.g., date/time, country/language, blood analyte unit of measurement, receiver mode, alert notification profiles, etc.).

More specifically, as indicated in the “My Settings” logical tree diagram 714a of FIG. 7B-1, the UI is configured to receive an “Ideal Range” (IR) via user-specified high and low thresholds for an ideal blood analyte level. For example, a blood analyte level that is within the ideal range or IR (i.e., a user's blood analyte level is less than or equal to the high threshold, and greater than or equal to the low threshold) is one that poses no risk to the user's health, whereas a blood analyte level that is above the high threshold or below the low threshold of the IR may be indicative of a potentially dangerous blood analyte concentration (which may, if left unaddressed, constitute a potential danger to the user's health). In many cases, a blood analyte level outside of the IR (i.e., in the BZRs or PR discussed infra) requires a user action to manage the blood analyte concentration. In one implementation where the sensor system is configured to detect blood glucose (BG) level, an exemplary high threshold for the IR is 130 mg/dl and an exemplary low threshold for the IR is 70 mg/dl, although these values are merely exemplary.

FIG. 7C illustrates an exemplary UI display 716a for setting a range of the IR via a capacitive touch screen input and display device (e.g., as might be found on a smartphone or dedicated receiver) according to one embodiment of the UI apparatus. FIG. 7C additionally includes an exemplary UI display 716b for setting the ranges of a daytime BZR. In the example of UI display 716b, the daytime BZR is expressed as a low range (less than the IR low threshold) and a high range (greater than the IR high threshold). In the aforementioned implementation, an exemplary high BZR value might be 140 mg/dl, while an exemplary low BZR value might be 60 mg/dl; above or below these values, respectively, the user might receive a “priority range” alert as described elsewhere herein.

FIG. 7D-1 illustrates exemplary UI displays 718 for setting parameters of the nighttime BZRs. In the example of UI display outputs 719a and 719b, the nighttime BZR is expressed as a low range (less than the IR low threshold) and a high range (greater than the IR high threshold), respectively. Additionally, the user can selectively turn each of the nighttime BZRs ON or OFF. In the aforementioned implementation where the sensor system is configured to detect BG level, an exemplary high BZR is 120-140 mg/dl, and an exemplary low BZR is 60-80 mg/dl for the night-time BZRs

In addition to receiving user-specified high and low ranges for the BZR, the UI is further configured to receive specified times for the nighttime BZR (i.e., “Night Settings”) and/or the daytime BZR. For example, as shown in UI display 721a in FIG. 7D-1, a user can set the nighttime BZR to be implemented in a range; e.g., from 11:00 pm to 8:00 am. Thus, in this example, the daytime BZR is implemented only after 8:00 am and before 11:00 pm. In other examples, the user can alternatively set specified times for implementation of the daytime BZR such as, e.g., in “Day Settings” (not shown), thereby implementing the nighttime BZR outside of the user-specified daytime period. In other examples, the UI can be configured to receive one or more additional specified time periods for implementation of a BZR, such as, an afternoon BZR.

The UI can be further configured to receive user-specified days for implementing alternative daytime and nighttime settings of the BZR (i.e., “Weekday” and “Weekend”) per the menu structure 714a of FIG. 7B-1. For example, as indicated in the UI display output 721a in FIG. 7D-1, a user can set the nighttime BZR to be implemented for weekdays (i.e., Monday through Friday), while the weekend (i.e., Saturday and Sunday) nighttime BZR is implemented 12:00 am to 10:00 am (as indicated in UI display 721b of FIG. 7D-1). In other examples, the user can select any desired day of the week or range of days of the week for association with a specified nighttime BZR, such as e.g., selecting

Saturdays through Tuesdays for a first specified nighttime BZR and Wednesdays through Fridays for a second specified nighttime BZR.

FIG. 7D-2 illustrates another embodiment of the exemplary blood analyte display for the receiver, wherein a unified or integrated single-screen approach is used, including setting the range limits for buffers in the context of both day and night. In this embodiment, a single display screen 731 is generated by the receiver, with multiple (here, eight) independent BG values 733 entered by the user for each of the selected contexts (e.g., four for daytime, and a corresponding four for nighttime in this example). This approach consolidates into one screen what in accomplished in several screens in other described embodiments. It will be appreciated, however, that other contexts and/or integrated display schemes may be utilized consistent with the disclosure, and even sub-contexts aggregated within a top-level context. For example, in one such scenario, the top-level “daytime” context may have “morning” and “afternoon” (temporal) sub-contexts, and/or activity-based sub-contexts such as e.g., “cross-fit training” or “morning walk”, especially in cases where the blood analyte level response or behavior may vary as compared to the user's normal ambulatory or waking state (e.g., sitting at their desk at work). Sub-sets of BG/BZ values can be specified for these sub-contexts as well if desired, on the same UI (not shown), so as to provide the user with their BG/BZ value profile for an entire part of a day, or even the entire day. As discussed supra, at step 706 of the method 700 of FIG. 7, the current blood analyte level is assessed and alerts are provided to the user as necessary. One exemplary implementation for step 706 of FIG. 7 (i.e., provision of soft alert and priority alert notifications based on the calculated current blood analyte level and the foregoing user-defined IR and BZRs) is shown in FIG. 7E. First, per steps 500/511 (discussed supra), a current blood analyte level is determined. The current blood analyte level is then compared to the user-specified BZR thresholds (such as e.g., presently implemented BZR thresholds dependent on current time and/or day of the week) at step 720. If the blood analyte level is greater than the high threshold of the high BZR (i.e., within the PR high range) or less than the low threshold of the low BZR (i.e., within the PR low range), a priority alert notification is generated and provided to the user via the receiver 400, 450 (and/or the parent platform 600) at step 722.

As discussed elsewhere herein, numerous alert notification mechanisms are contemplated for use with the presently described apparatus and methods. In general, a priority alert notification is one which is obvious and would be difficult for the user to ignore or sleep through. For example, a priority alert notification can include one or more of flashing light indicators, a strong haptic alert (e.g., a strong vibration), or a loud audible alert. In some examples, the user can select a strength (e.g., volume, degree of vibration, etc.) of the priority alert notification as well as the modality (or modalities) for delivery. Further, the user can select a progressive alert mechanism which increases in strength or intensity, and/or includes additional modalities over a duration of the notification (e.g., haptic, with addition of visual, then addition of audible alert notifications).

At step 724, it is determined whether the system has received user input regarding an action taken in response to the provided priority alert. For example, FIG. 7F illustrates exemplary UI displays 736 for notification of required action/attention and receiving user responses (i.e., input regarding an action taken by the user in response to the notification). Specifically, UI displays 737a and 737b are provided to the user (via a display device of the receiver 400, 450 and/or the parent platform 600) substantially simultaneously with the foregoing priority alert notifications in order to provide the user an opportunity to input an action taken in response to the alert. Iconic representations can also be used alone or in tandem with the shown displays; e.g., a flashing icon depicting a cookie or other food item indicative of the need to ingest a certain type of substance (e.g., fast or slow acting carbohydrate).

In the example of UI display 737a of FIG. 7F, a priority alert notification is provided in response to determination that the blood analyte level (i.e., 152 mg/dL) is within the high PR (i.e., greater than the high threshold of the high BZR). The UI display 737a enables the user to enter a user action taken in response to the high blood analyte level condition. In the example of UI display 737b, a priority alert notification is provided in response to determination that the blood analyte level (i.e., 60 mg/dL) is within the low PR (i.e., less than the low threshold of the low BZR). The UI display 737b enables the user to enter a user action taken in response to the low blood analyte level condition. In both examples, a list of user-selectable actions is displayed. In response to the priority alert notification, the user can select one (or more) of the user actions, such as e.g., intake of fast-acting carbohydrates, intake of slow-acting carbohydrates, and/or intake of insulin.

It is noted that, alternatively or additionally, the user action list can be provided to the user independent of an alert notification. For example, a user can set a timer or alert to send a reminder notification to the user via the UI indicating an action related to management of blood analyte level (such as e.g., intake of a medication). In another example, a user action list (such as e.g., UI displays 738 for user action input and reminders shown in FIG. 7G) can be selectively displayed in response to a user command (such as e.g., selection from a main menu) thereby enabling the user to enter a user action at any desired time. As depicted in the exemplary UI display 741 in FIG. 7G, the list may include, among other actions, intake of fast-acting carbohydrates, intake of slow-acting carbohydrates, intake of insulin, and entry of a manual BG reading (such as e.g., for calibration of the implanted sensor).

Exemplary UI displays 745a-745c shown in FIG. 7G enable a user to select and enter a time and date for a user action (e.g., intake of fast acting carbs, intake of slow acting carbs, and intake of insulin), while UI display 745d enables a user to enter or select a manual blood analyte reading (such as for e.g., calibration of the sensor system). It will be appreciated that the UI displays 745a-745c of FIG. 7G can additionally or alternatively be displayed and receive user input in response to selection of a user action from a list in one of UI displays 736 of FIG. 7F.

User action data (received via any of the foregoing UI displays shown in FIGS. 7F and 7G) is stored at the receiver 400, 450 and/or the parent platform 600 along with blood analyte level data. An exemplary UI display 743 (FIG. 7G) indicates a historical log of user actions.

Returning to FIG. 7E, if a user action input is received via the UI, a delay period is invoked per step 728 to allow the user's physiology to respond to the action prior to providing additional priority alert notifications. For example, after receiving a user action input, the receiver 400, 450 can invoke a delay period, such as e.g., a 5-minute delay period, so as to permit the action taken to take effect on the user's physiology. During that time the receiver 400, 450 may monitor the user's blood analyte level per step 730; however, even if the blood analyte level is in the PR (i.e., above the high BZR or below the low BZR), no additional priority alerts are provided throughout a duration of the delay period in the exemplary embodiment. Further, in some examples, even if the blood analyte level is above the IR high threshold or below the IR low threshold (i.e., within the high or low BZRs), no soft alert notifications are provided throughout a duration of the delay period. To this end, the delay period is selected so that even under a maximum anticipated rate of change scenario the user's BG level will not be expected reach a dangerous level during the duration of the delay, and in fact may be set dynamically by the computerized logic of the system based on e.g., ROC of BG level, absolute BG level (irrespective of relation to BZ, such as where the user's BG level is low enough such that seizure or other hypoglycemic effect is possible), and/or other factors.

After lapse of the delay period of step 728, the receiver 400, 450 continues to monitor/determine the blood analyte level (step 730), and additional alerts may be provided to the user as necessary.

Per step 726, if no user action input is received via the UI, a priority alert notification is sent to a parent or caretaker device. For example, a notification of a priority alert can be sent to a caretaker device (e.g., mobile device) indicating details regarding the blood analyte level condition, identification of the user (i.e., a patient or child of the caretaker), and/or a location of the user (such as e.g., via a GPS locator associated with the receiver 400, 450, association with a particular WLAN access point (AP) of known location, etc.). Thus, the priority alert notification provided to the caretaker device enables the caretaker to respond to and assist the user if they are incapacitated.

For example, if the user is unconscious due a blood analyte condition, the caretaker can locate and assist the user and/or contact paramedic services. In an alternate example, a priority alert notification can be sent to the caretaker device concurrently with the priority alert notification sent to the user device. In another alternate example, a priority alert notification can additionally or alternatively be sent directly to a health professional or health professional services (such as e.g., a hospital, paramedics, a doctor, a nurse, etc.) concurrently with the priority alert notification sent to the user device.

As discussed in greater detail infra, such functionality can also be combined with the “context” of the user, so as to properly structure the actions taken. For instance, if an accelerometer of the user's receiver (e.g., local receiver 400, dedicated receiver, or smartphone) indicates (i) substantial and continuing motion of the user contemporaneous with the detected PR-range blood analyte level, and (ii) the time of day is a time that the user is normally ambulatory (e.g., noon on a workday), the alert and monitoring regime may be structured such that repeated alerts are sent to the user only (no alerts sent to the caretaker device or health care provider), say at increasing frequency until the user responds appropriately. Conversely, if the user is detected via accelerometer to be non-ambulatory, and/or the time of day is evening or late at night (when the user might be asleep), then a more urgent regime is invoked, such as immediate notification of a caretaker, since the user may have lost consciousness (as indicated by lack of motion), and is unable to take corrective action.

Returning to step 720 of FIG. 7E, if the blood analyte level is less than the high threshold of the high BZR and greater than the low threshold of the low BZR (i.e., not within the PR), it is then compared to the user-specified IR thresholds (per step 732). If the current blood analyte level is less than the IR high threshold and greater than the IR low threshold (i.e., blood analyte level is within the IR), no alerts are generated, and the system continues to monitor and determine the blood analyte level (step 730). Alternatively, if the current blood analyte level is greater than the IR high threshold or less than the IR low threshold (i.e., blood analyte level is within one of the low BZR or the high BZR), a “soft” alert notification is provided to the user via the receiver 400, 450 (and/or the parent platform 600) at step 734.

In contrast to the priority alert notification of step 722, a soft notification per step 734 in one implementation is one which is non-obvious to others, and non-disruptive to the user. For example, a priority alert notification can include one or more of flashing light indicators, a haptic alert (e.g., a strong vibration), or an unmistakable audible alert, while a soft alert might include only one of the aforementioned modalities, at reduced intensity, such that only the user can (reasonably) perceive it. In some examples, the user can select a strength (e.g., volume, degree of vibration, etc.) of the soft notification as well as the modality for delivery. Further, the user can select a progressive alert mechanism which increases in strength and/or includes additional modalities over a duration of the notification (e.g., haptic, with addition of visual, then addition of audible alert notifications).

An exemplary sound profile model of volume, vibration, and screen display which can be utilized for creating soft and/or priority alert notifications is shown in FIG. 7N. In this example, a “Normal Mode” comprises vibration at a reasonably user-comfortable interval with display screen ON/OFF pulses, followed by an ascending audible alert to a prescribed value (e.g., 70% of the total volume), and continued vibration and screen pulses. An exemplary “Vibrate Only Mode” comprises vibration at a comfortable interval with display screen ON/OFF pulses. An exemplary “Loud Mode” comprises vibration at an initially comfortable interval with display screen ON/OFF pulses, followed by an ascending audible alert (with continued vibration and display screen pulses). An exemplary “Silent Mode” comprises suppression of all sounds and vibrations, which can be immediately overridden if a blood analyte level is within the PR. An exemplary “Night Mode” initially comprises similar conditions as the “Loud Mode” and additionally includes (after ascending the audible alert to e.g., 70% of the total volume) increased vibration frequency, turning the display screen to an ON state, and increasing the volume to 100%. An exemplary “Priority Mode” comprises the highest frequency of vibrations and volume, as well as turning the display screen ON.

In some embodiments, although not specifically shown in FIG. 7E, provision of soft alerts can additionally include provision of a UI display output, including a user response list substantially simultaneously with the soft notification, in order to provide the user an opportunity to input an action taken in response to the soft alert. For example, the UI display output 739a shown in FIG. 7F, a soft alert notification is provided in response to determination that the blood analyte level (i.e., 123 mg/dL) is within the high BZR (i.e., greater the high threshold of the IR). The UI display output 739a enables the user to enter a user action taken in response to the high blood analyte level condition (if desired).

In the example of UI display output 739b, a soft alert notification is provided in response to determination that the blood analyte level (i.e., 78 mg/dL) is within the low

BZR (i.e., less than the low threshold of the low IR). The UI display output 739b enables the user to enter a user action taken in response to the low blood analyte level condition (if desired). In both examples, a list of user-selectable actions is displayed. In response to the soft notification, the user can select one (or more) of the user actions, such as e.g., intake of fast-acting carbohydrates, intake of slow-acting carbohydrates, and/or intake of insulin. Further, a delay period may be implemented prior to sending additional soft notifications to allow the user's physiology to respond to the action.

It will be appreciated that the methodology for provision of alert notifications can include other additional or alternate features and steps. For example, in alternate or additional embodiments, the user can define rate-of-change (ROC) thresholds in a similar manner to setting IR and BZR thresholds. Thus, the exemplary method can additionally include providing alert notifications based on the current ROC. Specifically, in one example, if the blood analyte level is within the IR, but the ROC is above an ROC threshold (e.g., rapidly increasing or decreasing blood analyte level, which may be correlated with an undesirable underlying physiologic event or condition), an alert notification is generated and provided to the user.

In another example, user-defined ROC thresholds can be implemented in combination with the specified BZRs. In such an example, a soft alert notification may be provided to the user only if the blood analyte level is within the BZR and has an ROC greater than a specified ROC threshold (i.e., indicating the blood analyte level is rapidly increasing or decreasing). Further, if a user action input is received in response to the soft alert notification, additional soft alerts can provided only if the ROC is less than an ROC threshold (i.e., indicating the blood analyte level is substantially unresponsive to the user action).

Turning now to FIG. 7H, an exemplary method 706 for selectively displaying trend or other analyses of blood analyte level (see FIG. 7) is shown and described. As illustrated, the method 708 begins with receiving a user and/or caretaker request for data analysis at step 740, such as e.g., selection for data analysis from a general menu via the UI of the receiver 400, 600. In response to receiving the request for data analysis, the stored data (e.g., stored blood analyte data, stored user action data, etc.) is accessed (step 741), and analysis calculations are performed (step 742). Next, the calculated analysis data are converted to a graphic (or other) output format (step 743), which is output to a display device associated with the receiver 400, 450 (step 744) and/or the parent platform 600 for display to the user and/or caretaker (step 745).

In one exemplary implementation, unprocessed data from the sensor apparatus 200 is, on a periodic basis, scrambled then transmitted wirelessly from the sensor apparatus 200 to the receiver device 400, 450. Within the receiver device, the scrambled sensor data are unscrambled using an unscrambling algorithm, and stored in memory. These “raw” sensor data are then processed by various algorithms running on the receiver device processor(s) to yield a series of calculated blood glucose values. These algorithms incorporate adjustments derived from the periodic system calibrations performed by the user. The algorithms also filter the raw and processed data to reduce noise, and limit the perceived rate of change of the calculated blood glucose values to what is physiologically rational or possible (i.e., to avoid “non-phsysical” results). These calculated blood glucose values are then displayed to the user in numerical form, and are also used to graph the historical trends as points plotted on a time-axis chart, as a bar chart, and/or in tabular form. Additional algorithms of the receiver monitor the current calculated blood glucose values, and compare them to thresholds set by the user within certain system-defined constraints. When the calculated values exceed a specific threshold, the system initiates an alert to the user. The series of calculated blood glucose values is then further processed by yet other algorithms to deduce the relative trends in the blood glucose levels by comparing the current calculated value with previous calculated values. Those trends are then displayed to the user in the form of cognizable user interface elements such as arrows.

Independent of the system-generated blood glucose values and trending information, the user may also manually enter various “event” data, including for example manually entered blood glucose values (such as from an external calibration device), the performance of one or more insulin injections, and the consumption of carbohydrates. This manually entered log data is stored with time stamps in memory (and/or configured such that the user can enter the date/time of actual consumption, even if entered at a later time), and is displayed to the user in both tabular form and as icons in graphical form.

Exemplary UI display outputs 749 and 751 including graphical analyses of blood analyte level data are shown in FIGS. 7I and 7J, respectively. The UI display outputs 749 of FIG. 7I each include a line trend graphical analysis of blood analyte level data and user input data over a specified time period. Specifically, in one implementation, graphical analysis outputs 750a-750c show blood analyte level data and user input data over a 24 hour period, a 12 hour period, and a six hour period, respectively. Each of the graphical analysis outputs 749 includes a trend line 753 (indicated in the graphical analysis output 750a) which indicates blood analyte level with respect to the IR 755, the high BZR 757, and the low BZR 759.

As indicated the graphical analysis output 750a, the IR is defined by a high threshold at 761 and a low threshold at 763, which respectively correspond to a low threshold of the high BZR and a high threshold of the low BZR. Further, the daytime high

BZR is defined by a high threshold at 765, while the nighttime high BZR is defined by an increased high threshold at 769; and the daytime low BZR is defined by a low threshold at 767, while the nighttime low BZR is defined by a decreased low threshold at 771. Thus, as in the above described example, the high BZR is wider and the low BZR is narrower during a nighttime-designated period thereby decreasing sensitivity of the receiver/UI to a high BG level and increasing sensitivity of the receiver/UI to a low BG level while the user is sleeping.

According to the provision of alerts method 706 described supra, during periods of time where the trend line 753 is within the IR no alerts were generated. At points of time where the trend line 753 is above or below the IR within either of the low or high BZRs, soft alert notifications were generated. Additionally, at points where the trend line 753 is above the high BZR or below the low BZR, priority alert notifications were generated.

Also included in the graphical analysis outputs 749 are various recorded user action data received from the user via the UI in response to receipt of alert notifications and/or at the user's discretion. Specifically, in the graphical analysis output 750a, (i) in response to provision of a priority alert notification, a user input of fast-acting carbohydrate intake is indicated at 773; (ii) in response to provision of a priority alert notification, a user input of insulin intake is indicated at 775; (iii) at the user's discretion and/or in response to a notification, a user calibration input is indicated at 775; (iv) in response to provision of a priority alert notification, a user input of intake of slow-acting carbohydrates is indicated at 774; and (v) in response to provision of a soft alert notification, a user input of insulin intake is indicated at 772.

Turning to FIG. 7J, the UI display outputs 751 includes a bar graph analysis of blood analyte level data over a specified time period. Specifically, graphical analysis output 752a shows blood analyte level data over a one-week period, while graphical analysis output 752b shows blood analyte level data over a two-week period. Similar to the graphical analysis outputs 749, (as shown output 752a) graphical outputs 751 indicate a blood analyte level with respect to the IR 755, the high BZR 757, and the low BZR 759; however, blood analyte level is represented as bars 748, each representing a 6-hour period of a specific day. Each bar 748 indicates a range (i.e., a high and a low) of blood analyte level that occurred within the 6-hour period.

As indicated in graphical analysis output 752a, the IR is defined by a high threshold at 761 and a low threshold at 763, which respectively correspond to a low threshold of the high BZR and a high threshold of the low BZR. Further, the high BZR is defined by a high threshold at 765 and the low BZR is defined by a low threshold at 767. In contrast to the graphical output 749 discussed supra, alternate BZRs for nighttime and daytime are not reflected in graphical output 751 in order to accommodate visual representation of a larger data set (e.g., a one-week period of collected data vs. 24 hours of collected data). It will be appreciated that other exemplary bar graph outputs may include indication of nighttime and daytime BZRs.

According to the provision of alerts method 705 described supra (see FIG. 7), during periods of time where the bars 748 are within the IR no alerts were generated. During periods of time where the bars 748 are above or below the IR within either of the low or high BZRs, soft alert notifications were generated. Additionally, during periods of time where the bars 748 are above the high BZR or below the low BZR, priority alert notifications were generated.

It will be appreciated that graphical outputs 749 and 751 are merely two examples of data outputted in a user-cognizable form and the output of analytical blood analyte data via method 708 can include additional or alternate graphical and non-graphical data outputs for display at the UI. For example, UI display output 753 shown in FIG. 7K is a non-graphical analysis output. In the example of output 753, a table of data is displayed including a percentage of time that the user's blood analyte level was within the IR (i.e., 74%), as well as indicators of an identified time period (e.g., 10:00 am-1:00 am) when the user's blood analyte level was often high and a time period (e.g., 12:00 pm-1:00 pm) when the user's blood analyte level was often low.

Returning to FIG. 7H, at step 740 of method 706, a selection of an alternate analytical data output may be received from the user via the UI subsequent to the initial request. For example, FIG. 7L includes a UI display output 755 for a toggle menu. The toggle menu enables the user to scroll between and select the various display outputs such as e.g., the current blood analyte level display 712, the graphical outputs 749 and 751, and the non-graphical output 753. The toggle menu can further enable the user to scroll between current and historical data.

Turning now to FIG. 7M, in one embodiment, the method 707 of data error identification (see FIG. 7) first includes a determination of whether data have been received from the sensor per step 776.

If the data have not been received, the logic operative on the receiver 400, 450, 600 causes re-initiation of the relevant data interfaces (e.g., RF modem IC) per step 778, such as via a reset command invoked by the digital processor or CPU of the receiver.

This reset is performed to “reboot” the interface so as to (hopefully) cause clearing of any suspended states or similar operational software/firmware failures. It will be appreciated that step 778 may include various types of resets or other actions, whether serially or in parallel (e.g., reset CPU, reset implanted sensor via wireless command if so equipped, reset power supply, etc.) so as to attempt to correct the failure of data transfer from the implanted sensor to the receiver.

Also, per step 779, an integer counter (n) is incremented by one (1) for each reset, thereby enabling a user- or manufacturer-configurable number of attempts before an error flag is thrown, and immediate alert provided to the user (step 780), such as audible, haptic and visual alerts to indicate that the user may be in an unmonitored/un-alerted state with respect to BG level.

If data are received per step 776, then the received data are pre-processed by the receiver (step 781) so as to parse the data, generate appropriate protocol data packets or other data structures, etc. for subsequent processing, analysis, and logging in the memory of the receiver.

At step 782, the completeness of the pre-processed data is analyzed by the receiver logic; e.g., has the expected amount of data been received from the implanted sensor. For instance, with the exemplary sensor apparatus 200 described supra, four (4) detector element sets (one oxygen, one reference) are expected to produce a prescribed amount of data per sampling or communication interval; less than this prescribed amount may be indicative of e.g., a failed detector element or set within the implanted sensor 200. Accordingly, per step 783, logic on the receiver 400, 450, 600 may be invoked to attempt to identify the source of the incompleteness, such as through further analysis of the received data, the non-received or omitted data (i.e., by what is expected but missing from the data set, etc.).

If the received and pre-processed data set is determined to be complete per step 782, then per step 785, the calibration interval associated with the data is then determined.

The user will then be prompted via the UI (step 787) to conduct a calibration and enter data (step 788), the system using such received data per step 789 to refresh the system calibration. Notably, the user can either enter manual calibration data, and/or calibration data can be received from a calibration device (such as CSP 650 shown in FIG. 3B) at step 788. An exemplary UI display output for receiving manual calibration data is shown in FIG. 7G.

If the data received are within the prescribed interval per step 786, the accuracy of the data is next assessed per step 790. In one variant, this assessment comprises evaluation of the data for possible corruption or anomalous values. Hence, while all the data may have been received (complete) and within the calibration interval (fresh), it may nonetheless have been corrupted or be defective based on any number of possible error sources including for example: (i) bit errors in the wireless transmission mode that were not fixed by the wireless interface FEC; (ii) protocol errors during transmission, such as by the sensor apparatus or the receiver apparatus (e.g., field length over-runs, CRC errors, etc.); and/or (iii) non-physical data values (e.g., BG level values which are negative, impossibly high, exhibit non-physical rates of change from prior or later values, etc.). If such anomalies are detected, corrective measures are implement per step 792, which may include e.g., eliminating the suspected anomalous data from further consideration, re-booting the sensor and/or receiver (or specific components thereof), providing user alerts as to the presence of such data, calling out to a cloud-based processing entity (e.g., manufacturer's web server) for reporting and/or intervention (which may include a firmware update), etc.

At step 793, the processing of the data is then continued when all errors have been corrected, thereby assuring that it is at least: (i) complete; (ii) sufficiently fresh; and (iii) free from data/protocol errors, for subsequent generation of the BG level estimates.

It will also be appreciated that the logic of the method 707 may be configured so as to receive signaling or data from other processes or entities as to the status of various components, and/or data integrity. For instance, in one variant, the implanted sensor apparatus 200 itself may be equipped to detect incomplete/erroneous data prior to or concurrent with transmission. For instance, the communications protocol between the sensor 200 and receiver 400, 450, 600 may include error fields or flags.

Exhibit I hereto provides an exemplary embodiment of computer code for various of the UI and alert functions described above.

### Context Determination—

As referenced supra, certain embodiments of the present disclosure can combine the monitoring and/or alert regimes with data indicative of the “context” of the user, so as to properly structure the actions taken within the monitoring and/or alert regimes.

As a brief aside, modern personal electronics are increasingly equipped with a variety of sensors, as well as access to “artificial intelligence (AI)” such as cloud-based personal assistant functions (e.g., Google)Now®. Sensors may include for example accelerometers, position location devices (e.g., GPS or GLONASS receivers, or location detection via e.g., LTE/LTE-A base station or Wi-Fi AP association), temperature and pressure sensors, altimeters, biometric sensors (e.g., pulse rate, cardiac rhythm assessment algorithms, non-invasive blood pressure, “sweat” or perspiration sensors, calorimetric estimators), acoustic sensors, and yet others. These sensors may be co-located within a personal electronics form factor, such as a smartphone or smart watch, or Fitbit® type of personal monitoring/training device, as well as in other form factors (e.g., stand-alone pendants, skin patches, as part of athletic wear or clothing, etc.), including the receivers 400, 450 and parent platforms 600 previously referenced herein. Such sensors and AI functionality can be increasingly leveraged to determine user-specific context or state. Accordingly, embodiments of the present disclosure make use of data generated by such apparatus (whether in raw form, or having been pre-processed) to help guide blood analyte monitoring and alert regime configuration and operation, so as to inter alia, make the monitoring and alert regimes more accurate and intuitive (and reduce user frustration and annoyance from intrusive alert functions).

Specifically, in one implementation, the presence of one or more “context-capable” sensors or processes is evaluated prior to configuring or implementing the monitoring/alert regimes. As shown in FIG. 8, the method 800 includes first determining blood analyte level per step 801, such as using the techniques previously described herein with respect to the sensor apparatus 200 of FIG. 2.

Next, per step 802, the receiver context determination logic (e.g., computer software/firmware operative to run on the receiver and/or parent platform) determines if one or more prescribed context data sources are available, such as via accessing a register, receiving an inter-process signal or message, or yet other means. For instance, if an accelerometer of the receiver 400, 450 has been enabled, status indicators (e.g., register flags) for the accelerometer can be accessed and the status reflected within the receiver logic, or inter-process messages issued at higher layers of the protocol stack of the host device, such as from an accelerometer management routine or application to the computerized context logic process described herein.

One or more APIs (application programming interfaces) may be utilized to access/communicate with the various context data sources. Such APIs are especially useful for accessing higher layer processes, including cloud-based processes (e.g., third-party data providers). For instance, in one variant, the receiver context determination logic is configured to use the receiver's Wi-Fi or LTE interface to call a third-party cloud API such as one of the aforementioned personal assistant processes, thereby off-loading the context determination (or at least portions thereof) to the third-party. Consider the example where the user accesses the personal assistant application via the speech recognition interface of their smartphone, requesting the assistant to “wake me up at 6:00 am tomorrow.” In operation, such request is converted to textual data and transmitted wirelessly to the responsive server servicing the API call address (which is specified by the underlying application program running on the smartphone). Such API calls return, after processing by the cloud entity, formatted data which is responsive to the request. For instance, such wakeup call functionality of the example may cause the return of commands to the user's clock/alert application program on the smartphone to invoke the desired functionality, including perhaps appropriate audible profiles previously specified by the user. Such commands (or other data returned by the API call) are also indicative of a user's “sleep” context, which is very relevant to the user's BG level monitoring and alert regime, as previously discussed herein.

Similarly, a user's status notification via the assistant (or even social media applications) can be used by the context determination algorithms as inputs. For example, if the user updates their assistant or social media account for “at lunch”, this information may be used by the context determination algorithms to configure the monitoring and alert regimes for an ambulatory, daytime mode (assuming consistent with any temporal inputs) where ingestion of fast- and/or slow-acting carbohydrates may occur.

If no context data sources are available per step 802, the logic reverts to step 803, wherein the monitoring/alert regime of FIG. 7E is implemented (i.e., context-less monitoring).

If context data sources is/are available, then descriptive information of the source(s) is obtained per step 804. For instance, the receiver context determination logic may be configured to associate ID codes to particular sources, such that the logic can appropriately utilize any data obtained from the source. Such identification of the source may also be intrinsic in nature; i.e., by virtue of receiving data of a certain type/format/frequency, or via a certain software port or interface, it is known or presumed to be associated with a certain type of source.

Per step 805, the receiver logic uses the source ID data to access a source ID database maintained by the context determination logic, so as to reconcile the types of sources available. Per step 806, if the source ID(s) is/are found within the database (ie., are recognized by the context determination logic), the data sets associated with the source IDs (i.e., whether by local data access, local or “cloud” API call, or other) are accessed per step 809, and the data supplied to the context determination algorithm(s) for context determination (steps 810, 811). Ultimately, the output of the context determination logic is supplied to the monitoring/alert regime logic (step 812) to enable proper user-specific configuration of monitoring and alerts based on the determined context.

As one example, a given receiver may have available to it, based on the respective source IDs: (i) accelerometer data; (ii) GPS position or location data; and (iii) temporal or time data. Based thereon, the context determination logic of the receiver can determine the user's ambulatory context, such as from a prescribed set of possibilities (e.g., awake and ambulatory; awake but stationary; sleeping). For instance, if an accelerometer of the user's receiver (e.g., local receiver 400, dedicated receiver, or smartphone) indicates (i) substantial and continuing motion of the user contemporaneous with a detected priority-range blood analyte level, and (ii) the time of day is a time that the user is normally ambulatory (e.g., noon on a workday), and the GPS location corresponds to their work location (or a location outside of their home), the alert and monitoring regime may be structured such that repeated alerts are sent to the user only (no alerts sent to the caretaker device or health care provider), e.g., at increasing frequency until the user responds appropriately. Conversely, if the user is detected via accelerometer to be non-ambulatory, and/or the time of day is evening or late at night (when the user might be asleep), and the location is the user's home, then a more urgent regime is invoked, such as immediate notification of a third party, since the user may have lost consciousness (as indicated by lack of motion), and is unable to take corrective action.

FIG. 8B illustrates one exemplary embodiment of the local receiver 400 of FIG. 3, configured to implement the foregoing methodologies of FIGS. 8 and 8A. Specifically, in the illustrated implementation, the receiver 400 comprises a small form-factor wrist worn device having, inter alia, the computerized context determination algorithms (part of the illustrated logic 408), accelerometer 496, GPS receiver 497, and non-invasive tonometric blood pressure sensor and heart rate (HR) sensor 498. Moreover, the computerized context algorithms include logic capable of indirectly accessing one or more APIs associated with a cloud based assistant process 495, such as via the parent platform 600 and its indigenous assistant application. Alternatively, the local receiver 400 can be configured with its own logic and assistant application such that direct access of the cloud-based process 495 can be accomplished (e.g., via the communications interface 414 directly to a base station or AP).

In operation, the computerized context determination algorithms periodically poll or otherwise access data generated by the indigenous sensors 496, 497, 498 to obtain respective values for acceleration, location, and blood pressure/heart rate, such as via calls to respective APIs associated with applications running on the receiver 400 associated with each of the sensors. These data sets are used as inputs to the context determination algorithms, as well as any data accessed from the personal assistant process 495.

Notably, by placing the sensors 496, 497, 498 or others directly on the local receiver 400 (as opposed to the parent 600 or other platform), the illustrated embodiment of FIG. 8B ensures that the acceleration, location and NIBP/HR data are: (i) associated with the user being monitored directly, and (ii) always available, since the local receiver 400 is designed to constantly be worn by the user for BG monitoring and alerts. However, as indicated previously, one or more of the sensors may be disposed on other platforms.

In one such variant, data is only accessed from such off-local sensors when it can be affirmatively ascertained that the local receiver and sensor platform are within sufficient proximity of one another (e.g., via near-field, Bluetooth, Zigbee, or other short range wireless modality) so as to ensure it is being carried by the user, and/or that the sensor is producing relevant and accurate data (e.g., the HR process is able to resolve a heart rate from a living being).

Moreover, it will be appreciated that one or more of the sensors 496, 497, 498 or others can be placed on the implanted sensor apparatus 200, with data generated by the sensors 496, 497, 498 transmitted (whether in raw or processed form) via the wireless interface of the apparatus 200 to e.g., a local receiver 400, 450 or parent platform 600 for use thereby; i.e., for input to the context determination algorithms/application program. In this fashion, there is 100% surety that the sensor data are in fact associated with a particular user and always available (barring equipment failure). However, such sensors and any processing and/or transmission thereof may cause additional electrical power consumption within the implanted sensor apparatus 200, and hence any such effects should be considered so as to maximize implanted longevity of the sensor apparatus 200.

The foregoing algorithms and hardware/firmware configurations can also be used to generate confidence data for use by the monitoring/alert regime algorithms to structure the appropriate regime. For instance, if the context determination algorithms have sufficiently detailed and/or redundant data sources, the confidence that the user is in fact within the determined context or state is higher than if such determination is based on lesser numbers or detailed sources. Horizontal posture and little or no motion shown via the accelerometer 496, constant GPS position per the GPS receiver 497, and slow, steady HR and (characteristically) low systolic/diastolic BP, as well as the aforementioned “wake me up at 6:00 am tomorrow” command to the personal assistant 495 would produce a high confidence that the user is in fact asleep. However, any one of those data sources taken alone could in fact produce an erroneous context determination; i.e., the user may be sitting reading (and hence no motion, low HR/BP, no change in GPS location, and merely being proactive by setting the alert early before actually retiring to bed).

Accordingly, the ROC and proximity of BG level to a particular alert level or BZR may be used by the monitoring and alert algorithms, as well as the confidence data generated by the context determination algorithm, to structure the monitoring/alert regime. For instance, if there was a high negative ROC value for BG, and the value was proximate the lower BZR (indicating a potential impending hypoglycemic condition), a relatively low confidence level on context (e.g., that the user was awake) might cause the monitoring/alert regime to be structured more conservatively than if the confidence was higher, and/or the ROC/BG level were not potentially problematic.

### Exemplary Use Cases

Lastly, various use-cases or scenarios for utilization of the receiver and UI are schematically illustrated in FIGS. 8A-8D. Specifically, a “Running High” scenario (i.e., blood analyte levels in the high BZR and/or PR) is depicted in FIG. 8A, a “Within Ideal Range” scenario (i.e., blood analyte levels in IR) is depicted in FIG. 8B, a “Running Low (night mode)” scenario (i.e., blood analyte levels in the low BZR and/or PR at a nighttime designated period) is depicted in FIG. 8C, and a “Priority Alert (low)” scenario (i.e., i.e., blood analyte levels in the low BZR and/or PR) is depicted in FIG. 8D. It will be appreciated that the scenarios shown in FIGS. 8A-8D are merely exemplary scenarios and a myriad of other use-cases may be produced or encountered via utilization of the apparatus and methods described herein.

It will be recognized that while certain embodiments of the present disclosure are described in terms of a specific sequence of steps of a method, these descriptions are only illustrative of the broader methods described herein, and may be modified as required by the particular application. Certain steps may be rendered unnecessary or optional under certain circumstances. Additionally, certain steps or functionality may be added to the disclosed embodiments, or the order of performance of two or more steps permuted. All such variations are considered to be encompassed within the disclosure and claimed herein.

While the above detailed description has shown, described, and pointed out novel features as applied to various embodiments, it will be understood that various omissions, substitutions, and changes in the form and details of the device or process illustrated may be made by those skilled in the art without departing from principles described herein. The foregoing description is of the best mode presently contemplated. This description is in no way meant to be limiting, but rather should be taken as illustrative of the general principles described herein. The scope of the disclosure should be determined with reference to the claims.

What is claimed is:

1. A method of monitoring a blood analyte level of a living being using a blood analyte sensing apparatus, the method comprising:
receiving blood analyte data from a blood analyte sensing apparatus;
evaluating the received data, the evaluating comprising calculating at least one parameter; and
based at least on the at least one parameter, configuring at least one user interface alert function.

2. The method of claim 1, wherein the received blood analyte data are generated periodically by a fully implanted oxygen-based blood glucose sensing apparatus.

3. The method of claim 2, wherein the calculating comprises calculating: (i) rate of change of blood glucose level; and (ii) blood glucose level; and
wherein the configuring at least one user interface alert function comprises establishing upper and lower buffer zones within which only soft alerts to the user are provided.

4. The method of claim 3, wherein the establishing upper and lower buffer zones within which only soft alerts to the user are provided comprises establishing an upper zone which is asymmetric in range with the lower zone.

5. The method of claim 4, wherein the range of the lower zone is smaller than the range of the upper zone, and the method further comprises alerting the user more rapidly for a given rate of change (ROC) when in the lower zone than in the upper zone, so as to more rapidly alert the user to the possibility of a hypoglycemic event.

6. The method of claim 4, wherein the provision of soft alerts comprises providing only non-audible alerts selected from the group consisting of (i) haptic alerts via a haptic apparatus of a receiver worn by the user, and (ii) visual alerts on a display device of the receiver.

7. The method of claim 1, wherein:
the calculating comprises calculating: (i) rate of change of blood analyte level; and (ii) blood analyte level; the configuring at least one user interface alert function comprises establishing upper and lower buffer zones within which only soft alerts to the user are provided; and
the method further comprises providing hard alerts when blood analyte level falls outside of either the upper or lower buffer zones.

8. The method of claim 1, further comprising receiving data from a sensor apparatus, the data indicative of at least one user context; and
establishing upper and lower buffer zones;
evaluating the data using a computerized process to identify a context; and
wherein the establishing upper and lower buffer zones comprises establishing at least one of the upper and lower zones based at least on the identified context.

9. The method of claim 8, further comprising:
accessing data indicative of a most recent calibration event;
calculating a value using at least (i) the accessed data indicative of a most recent calibration event, and (ii) data indicative of at least one of a current date and/or current time, the calculated value indicative of at least one of a date difference and/or time difference; and
wherein the establishing upper and lower buffer zones comprises establishing at least one of the upper and lower zones based at least on the identified context and the calculated value.

10. The method of claim 1, further comprising:
accessing data indicative of a most recent calibration event;
calculating a value using at least (i) the accessed data indicative of a most recent calibration event, and (ii) data indicative of at least one of a current date and/or current time, the calculated value indicative of at least one of a date difference and/or time difference; and
wherein the establishing upper and lower buffer zones comprises establishing at least one of the upper and lower zones based at least on the calculated value.

11. The method of claim 10, wherein the establishing at least one of the upper and lower zones based at least on the calculated value comprises determining a range of the at least one of the upper and lower zones that is inversely proportional to a magnitude of the calculated value.

12. The method of claim 10, wherein the establishing at least one of the upper and lower zones based at least on the calculated value comprises determining a range of the at least one of the upper and lower zones that is: (i) a constant value until a prescribed threshold of the calculated value is reached, and (ii) a value lower than the constant value after the threshold is reached or exceeded.

13. The method of claim 2, wherein the calculating comprises calculating at least one of: (i) rate of change of blood glucose level; and (ii) blood glucose level; and wherein the configuring at least one user interface alert function comprises:
evaluating the calculated at least one (i) rate of change of blood glucose level; and (ii) blood glucose level relative to established upper and lower buffer zones; and
configuring one or more soft alerts to the user based at least on said evaluating.

14. The method of claim 13, wherein the established upper and lower buffer zones comprise an upper zone which is asymmetric in range with the lower zone.

15. The method of claim 14, wherein the range of the lower zone is smaller than the range of the upper zone, and the method further comprises alerting the user more rapidly for a given rate of change (ROC) when in the lower zone than in the upper zone, so as to more rapidly alert the user to the possibility of a hypoglycemic event.

16. Apparatus for use with an implanted blood analyte sensing device, comprising:
wireless receiver apparatus configured to receive wireless signals from the blood analyte sensing device, the wireless signals encoding data relating to levels of said blood analyte;
data processing apparatus in data communication with the wireless receiver apparatus and configured to utilize said encoded data to determine a blood analyte level;
an electrical power source configured to supply electrical power;
indicator apparatus in communication with the data processing apparatus and electrical power source, the indicator apparatus configured to indicate to a user the determined blood analyte level; and
at least one computer program operative to run on the data processing apparatus and configured to selectively determine at least one of an upper buffer zone and lower buffer zone, the at least one buffer zone comprising a range of blood analyte level values within which a first alert regime is applied, and outside of which at least a second alert regime is applied, the second alert regime configured to be more intrusive to the user than the first regime.

17. The apparatus of claim 1, wherein said implanted blood analyte sensing device comprises at least one oxygen-based sensor, and said apparatus is configured to operate without communication with a parent platform or external calibration for at least one (1) week.

18. A method of monitoring a blood analyte level of a living being using a blood analyte sensing apparatus, the method comprising:
determining a context of the user; and
based on the determined context, implementing a context-specific monitoring and alert regime.

19. The method of claim 18, wherein the determining a context comprises at least:
receiving data from an accelerometer apparatus of the blood analyte sensing apparatus; and
evaluating the received data to identify one or more movement patterns associated with a user behavior; and
based at least on the identification of the one or more patterns, determining the context.

20. The method of claim 19, wherein the identification of the one or more movement patterns comprise comparison of at least a portion of the received data to one or more pre-stored templates.

21. The method of claim 18, wherein:
the blood analyte sensing apparatus comprises: (i) an implanted blood analyte sensor; and (ii) a local receiver apparatus in wireless communication with the implanted sensor; and the determining a context comprises at least using a personal assistant application program of a computerized device with which the local receiver apparatus is in data communication to access a network-based application programming interface (API) to obtain context data, the context data used in the determining the context.

22. A method of monitoring a blood analyte level of a living being using a blood analyte sensing apparatus comprising an implanted sensor apparatus and a receiver apparatus, the method comprising:
receiving at the receiver apparatus blood analyte data from the implanted sensor apparatus;
evaluating the received blood analyte data, the evaluating comprising calculating at least one parameter;
based at least on the at least one parameter, configuring at least one user interface alert function, the configuring the at least one alert function comprising establishing a first buffer range of blood analyte levels above a normal range, and a second buffer range of blood analyte levels below the normal range;
causing only a first type of alert to be provided to the user when the blood analyte level indicated by the at least one parameter falls within the first or second buffer ranges; and
causing only a second type of alert to be provided to the user when the blood analyte level indicated by the at least one parameter falls outside of the first and second buffer ranges and the normal range; wherein the causing only a first type of alert to be provided to the user when the blood analyte level indicated by the at least one parameter falls within the first or second buffer ranges, and only a second type of alert to be provided to the user when the blood analyte level indicated by the at least one parameter falls outside of the first and second buffer ranges and the normal range, reduces the frequency of the second type of alerts provided to the user via the at least one user interface over that provided without use of the first and second buffer ranges.

22. The method of claim 22, wherein;
the blood analyte comprises blood glucose;
the first alert type comprises a soft alert comprising no intentional audible sounds, and the second alert type comprises a hard alert comprising at least an audible sound; and
the first buffer range is greater in magnitude than the second buffer range so as to provide more conservative alerts of a low blood glucose level than a high blood glucose level.

24. A method of monitoring a blood analyte level of a living being using a blood analyte sensing apparatus comprising an implanted sensor apparatus having at least prescribed stability of blood analyte level measurement over a prescribed period of time, and a receiver apparatus, the method comprising:
receiving at the receiver apparatus blood analyte data from the implanted sensor apparatus, the blood analyte data associated with at least one first time reference;
determining a time reference associated with a last calibration of the blood analyte sensing apparatus;
determining a difference between the at least one first time reference and the time reference associated with the last calibration;
evaluating the received blood analyte data, the evaluating comprising calculating at least (i) a blood analyte level, and (ii) a rate-of-change (ROC) of the blood analyte level; and
configuring at least one user interface alert function, the configuring the at least one alert function comprising establishing a first buffer range of blood analyte levels above a normal range, and a second buffer range of blood analyte levels below the normal range, a magnitude of at least one of the first range and second range being selected based at least on the (i) the difference, (ii) the calculated blood analyte level, and (iii) the calculated ROC.
